SEQUENCE LISTING (20 sequences)

SEQ ID NO: 1 - DNA, 43 bp, synthetic construct
  misc_feature 1..43: note = Rail 1
  source 1..43: mol_type = other DNA
cctatcgtgttttgtcgctgaccatcgtatcgcttcgtctatg

SEQ ID NO: 2 - DNA, 41 bp, synthetic construct
  misc_feature 1..41: note = Rail 2
  source 1..41: mol_type = other DNA
ctgagtgaatgagctctacactgcagtaccaccgttagtca

SEQ ID NO: 3 - DNA, 21 bp, synthetic construct
  misc_feature 1..21: note = Staple 1
  misc_feature 10..11: note = /iSp18//iSp18/ insertion point
  source 1..21: mol_type = other DNA
attcactcagcatagacgaag

SEQ ID NO: 4 - DNA, 30 bp, synthetic construct
  misc_feature 1..30: note = Staple 2
  misc_feature 15..16: note = /iSp18//iSp18/ insertion point
  source 1..30: mol_type = other DNA
gacaaacacgataggtgactaacggtccag

SEQ ID NO: 5 - DNA, 11 bp, synthetic construct
  misc_feature 1..11: note = Blck A1
  source 1..11: mol_type = other DNA
cgatacgatgg

SEQ ID NO: 6 - DNA, 10 bp, synthetic construct
  misc_feature 1..10: note = Blck B1
  source 1..10: mol_type = other DNA
tgtagagctc

SEQ ID NO: 7 - DNA, 10 bp, synthetic construct
  misc_feature 1..10: note = Blck A2
  source 1..10: mol_type = other DNA
tcagcgacaa

SEQ ID NO: 8 - DNA, 10 bp, synthetic construct
  misc_feature 1..10: note = Blck B2
  source 1..10: mol_type = other DNA
ggtactgcag

SEQ ID NO: 9 - DNA, 31 bp, synthetic construct
  misc_feature 1..31: note = A1
  misc_feature 8..9: note = /iSp18/ insertion point
  misc_feature 20..21: note = /iSp18/ insertion point
  source 1..31: mol_type = other DNA
ctttgttcagacaatgtagccgatacgatgg

SEQ ID NO: 10 - DNA, 29 bp, synthetic construct
  misc_feature 1..29: note = B1
  misc_feature 10..11: note = /iSp18/ insertion point
  misc_feature 20..21: note = /iSp18/ insertion point
  source 1..29: mol_type = other DNA
agtagagctcgaaacccagcgatgattcc

SEQ ID NO: 11 - DNA, 28 bp, synthetic construct
  misc_feature 1..28: note = A2
  misc_feature 10..11: note = /iSp18/ insertion point
  misc_feature 18..19: note = /iSp18/ insertion point
  source 1..28: mol_type = other DNA
tacattgtctggtgaacctcagcgacaa

SEQ ID NO: 12 - DNA, 14 bp, synthetic construct
  misc_feature 1..14: note = B2
  misc_feature 8..9: note = /iSp18/ insertion point
  source 1..14: mol_type = other DNA
tgttgctcgctggg

SEQ ID NO: 13 - DNA, 32 bp, synthetic construct
  misc_feature 1..32: note = Bridge
  misc_feature 22..23: note = /iSp9/ insertion point
  source 1..32: mol_type = other DNA
aggggttcaccgagcaacattcggtactgcag

SEQ ID NO: 14 - DNA, 49 bp, synthetic construct
  misc_feature 1..49: note = A3
  misc_feature 8..9: note = /iSp18/ insertion point
  misc_feature 16..17: note = /iSp18/ insertion point
  misc_feature 27..28: note = /iSp18/ insertion point
  source 1..49: mol_type = other DNA
ctttgttcagacaatgcgatacgatgggctacattgtctgctgggtttc

SEQ ID NO: 15 - DNA, 27 bp, synthetic construct
  misc_feature 1..27: note = B3
  misc_feature 10..11: note = /iSp18/ insertion point
  misc_feature 18..19: note = /iSp18/ insertion point
  source 1..27: mol_type = other DNA
agtagagctcaacccagcgatgattcc

SEQ ID NO: 16 - DNA, 22 bp, synthetic construct
  misc_feature 1..22: note = Input 3
  source 1..22: mol_type = other DNA
gaaacccagcagacaatgtagc

SEQ ID NO: 17 - RNA, 22 nt, synthetic construct
  misc_feature 1..22: note = Input 2
  modified_base 1: mod_base = OTHER; note = 5'-phosphorylation
  source 1..22: mol_type = other RNA
gaatgttgctcggtgaacccct

SEQ ID NO: 18 - RNA, 23 nt, synthetic construct
  misc_feature 1..23: note = Input 1
  modified_base 1: mod_base = OTHER; note = 5'-phosphorylation
  source 1..23: mol_type = other RNA
agctacattgtctgctgggtttc

SEQ ID NO: 19 - DNA, 30 bp, synthetic construct
  misc_feature 1..30: note = MB1
  modified_base 1: mod_base = OTHER; note = 56-TAMN(NHS Ester)
  modified_base 30: mod_base = OTHER; note = 3'-BHQ2 (Black Hole Quencher-2)
  source 1..30: mol_type = other DNA
cctggaatcatcgaacaaagcacagccagg

SEQ ID NO: 20 - DNA, 26 bp, synthetic construct
  misc_feature 1..26: note = MB2
  modified_base 1: mod_base = OTHER; note = 56-FAM (5(6)-Carboxyfluorescein)
  modified_base 26: mod_base = OTHER; note = 3'BHQ_1 (Black Hole Quencher-1)
  source 1..26: mol_type = other DNA
ccaggcccagcagacaatgtacctgg

(19) United States
(12) Patent Application Publication
(10) Pub. No.: US 2025/0340927 A1
(43) Pub. Date: Nov. 6, 2025

(54) MODULAR DNA LOGIC GATE UNITS FOR MOLECULAR COMPUTATION

(71) Applicant: UNIVERSITY OF CENTRAL FLORIDA RESEARCH FOUNDATION, INC., Orlando, FL (US)

(72) Inventors: Dmitry Kolpashchikov, Orlando, FL (US); Andrea Bardales, Orlando, FL (US)

(21) Appl. No.: 19/067,346

(22) Filed: Feb. 28, 2025

Related U.S. Application Data
(60) Provisional application No. 63/559,370, filed on Feb. 29, 2024.

(51) Int. Cl. C12Q 1/6816; C12Q 1/6886

(52) U.S. Cl. CPC C12Q 1/6816; C12Q 1/6886; C12Q 2600/158; C12Q 2600/178

(57) ABSTRACT

Herein, based on Boolean operators, DNA logic gate complexes are constructed to obtain True (fluorescence signal recovery) or False (fluorescence signal quenching) value using a molecular beacon acting as output. A DNA logic gate complex is composed of a DNA board and at least two modular logic units of YES and/or NOT gate. First, the DNA board is designed to accommodate individual YES and/or NOT modular logic units. An OR logic gate is composed of the DNA board and at least two YES modular logic units, and when there are multiple YES modular logic units, they are arranged in a parallel manner. A NAND logic gate is composed of a DNA board and at least two NOT modular logic units, and when there are multiple NOT modular logic units, they are arranged in a parallel manner. An IMPLY logic gate comprises at least one YES modular logic unit and at least one NOT modular logic unit as well as the DNA board. The designed DNA logic gate complexes were tested with biomarker miRNAs of hepatocellular carcinoma and successfully computed True/False fluorescence outcomes, exhibiting great potential for applications in the field of diseases diagnosis.

### GOVERNMENT FUNDING SUPPORT

The invention was made with government support under grant numbers SHF-1907824 and 2226021 awarded by the National Science Foundation. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 21, 2025, is named “10669-417US1.xml” and is 35,380 bytes in size. The sequence listing contained in this .XML file is part of the specification and is hereby incorporated by reference herein in its entirety.

### BACKGROUND

### 1. Field of the Invention

The invention relates to the field of cancer diagnosis by detecting biomarker nucleotides, and in particular to a method using DNA logic gates such as OR, NAND and IMPLY logic gates.

### 2. Background of the Invention

Early diagnosis of cancer is very important to increase the chance for successful treatment and survival of a patient as well as to reduce costs for care. For cancer diagnosis, biopsies have been commonly used, but this method is invasive and uncomfortable for patients. Therefore, various kinds of blood-based cancer assays have been developed for the detection of biomarkers such as protein, microRNA, circulating DNA, and methylated DNA. However, there are limiting factors such as specificity, overdiagnosis, and costs for the healthcare system. Thus, detection tools with high selectivity and specificity, and high-throughput at low-cost will improve early cancer detection.

Recently, microRNAs (miRNAs) have been considered as efficient biomarkers for the diagnosis of cancers as well as other nervous system-, cardiovascular system-, metabolic- and inflammatory/immune diseases. MicroRNAs are a class of small RNA molecules with a length of 19 to 24 nucleotides, and they are known to play important roles in posttranscriptional gene regulation in development, differentiation, and control of cell cycle. In cancer, some miRNA expressions are upregulated and others downregulated, and such changes can be the cause or result of cancer. For profiling miRNA expression levels, real-time PCR, microarrays, and RNA sequencing have been used, however, these methods are expensive and time-consuming, and accuracy is also in question.

Biotechnological tools oriented toward nucleic acid biomarkers have attracted the attention of the scientific community, thus leading to increasing efforts in their development. On the path toward personalized medicine, multiple nucleic acid sequences must be analyzed for precise diagnosis and proper treatment. Such nucleic acid sequences become inputs in the decision-making of molecular devices that produce a diagnostic and/or therapeutic output. These inputs can undergo complex algorithms (similar to those executed by electronic computers) for their analysis.

Although electronic computers are made from semiconductors, other materials can be used to build computers. Individual molecules and atoms have been proposed as building units for molecular computers. Efforts have been made to build molecular computers out of DNA since it is a compatible material for directly computing nucleic acid biomarkers. By using the principles of digital computing, DNA molecular computers are capable of multiplex parallel recognition of biomarkers (e.g., microRNA (miR)). The advantages of building molecular computers from DNA are their biocompatibility, affordability, and chemical stability.

In recent years, various DNA logic gates have been introduced for more accurate and faster detection of miRNA expression profiling. In most DNA logic gates, the scheme is based on the concept of DNA origami, in which a long single stranded DNA is aided by multiple short single stranded DNAs (staples). For example, in an AND logic gate, to generate output signal indicating miRNA binding to the gate, which is generally fluorescence- or colorimetric signal, an analyte miRNA (input) binding to the first toehold region triggers strand displacement to release a short single stranded DNA fragment (first output), and the released strand diffuses to a next toehold region for a next strand displacement, and these steps are repeated in a serial manner until the final output probe opens or are released, and sometimes deoxyribozyme sequence is inserted into the gate design for the release of the output probe.

Such complexity in DNA nanostructure design is a kind of double-edged sword in that it has the advantage of allowing it possible to attach multiple short fragments of any desired nucleotide sequence in either orientation in a serial manner. However, it also has disadvantages, e.g., small production scale, high error rate, and laborious sequence design for multiple inputs if multiple inputs are allowed.

### SUMMARY

To overcome the aforementioned disadvantages, herein novel designs of DNA logic gates, i.e., OR, NAND, and IMPLY logic gates, are introduced. The proposed design can accommodate multiple YES and/or NOT modular logic units in a parallel manner, and simultaneously recognize multiple input miRNAs.

These gates are based on a similar structure, which comprises a DNA scaffold structure functioning as a hybridization board, and at least two modular logic units of YES gate and/or NOT gate, to put out True (fluorescence signal recovery) or False (fluorescence signal quenching) result according to Boolean True/False logic computation.

Boolean logic gates are the most basic components in electronic computers [1]. A set of AND, OR, and NOT gates is a well-known, functionally complete set in digital computers [1]. This set has attracted attention because of its universality—the ability to achieve any other logic functions by integrating multiple units of this limited set [2]. This modular and scalable approach enables the easy design and cost-efficient manufacturing of computational circuits.

A YES gate modular logic unit produces a True (high fluorescence) output in the presence of an input and a False (low fluorescence) output in the absence of an input (FIG. 2a). A NOT gate modular logic unit is the inverter of YES logic (FIG. 2b). In digital computing, neither the combination of YES and NOT gates, nor NOT gates alone, have ever been reported to comprise a functionally complete set of gates.

Boolean logic gates made of small organic molecules [6], proteins [7], and nucleic acids [8] have been reported. It is believed that such gates can be used to build computational circuits that are smaller, consume less energy, and are capable of multiple parallel computing [8,9]. Furthermore, logic gates made of DNA and RNA can be used as molecular tools for diagnosis and therapy [10].

In this disclosure, DNA logic gates connected to each other via DNA four-way junction (4J) structures [11,12] have been developed. The gates recognize nucleic acid sequences as inputs and induce a new sequence arrangement by bringing two oligonucleotide fragments into proximity, which are the output binding sequence of the 4J gates. The new output binding sequence can be conveniently detected by a molecular beacon (MB) probe—a short nucleotide labeled with a fluorophore, which is masked by a quencher when the MB is a hairpin structure, i.e., unbound and closed free form [13]. The change in fluorescence generated from the open/closed MB probe can be correlated to the binary response (1 and 0) as in digital computing.

The YES or NOT modular logic unit is composed of a pair of strands, arbitrarily A strand and B strand. As inputs, various oligonucleotides can be contemplated, in particular biomarker miRNAs. As output, fluorescence generated from a molecular beacon is displayed.

FIG. 2a illustrates the functional mechanism of a 4J YES Boolean logic gate with the output sequence (strands A₁+B₁) triggering the MB₁ probe opening after input recognition. In the 4J NOT gate (FIG. 2b), strands A₂ and B₂ are brought together by a DNA “bridge”, which stabilizes their hybridization with the MB₂ probe in the absence of the input, thus enabling a high fluorescent signal (digital 1 or True). In this setting, the 4J NOT gate follows the NOT logic truth table by giving a functional output (output 1 or True) for input 0 (absence/low or False). The addition of an oligonucleotide input decomposes the 4J structure by hybridizing to the bridge fragment and triggering the dissociation of A₂ and B₂, which results in the release of the MB₂ probe. This causes MB₂ to fold itself as a hairpin and to exhibit low fluorescence (digital 0 or False).

To facilitate communication between YES modular logic units or NOT modular logic units, the gates are spatially localized on a DNA board, named here a DNA board, which is composed of two rail strands (Rail 1 and Rail 2) and two staple strands (Staple 1 and Staple 2) (FIG. 2c). The DNA board contains a single-stranded (ss) DNA region that serves as a flexible hybridization board for the integration of multiple DNA modular logic units, which allows for DNA circuits to be built.

The DNA logic gates (e.g., OR gate, NAND gate, and IMPLY gate) can be assembled in a simple way: mixing oligonucleotides (i.e., DNA board oligonucleotides and modular logic unit oligonucleotides), denaturing the mixture at a high temperature, and letting the oligonucleotides renature at room temperature, and in experiments, they assembled with high assembly efficiency. Besides, the DNA boards can be easily extended without sequence changes.

An OR gate is a DNA logic gate complex comprising at least two YES modular logic units parallelly integrated into a DNA board, and is TRUE, i.e., generating fluorescence, when any of input binding regions on the modular logic units of the gate binds to an analyte having a complementary nucleotide sequence.

A NAND gate is a DNA logic gate complex comprising at least two NOT modular logic units parallelly integrated into a DNA board, which has a bridge strand (i.e., an auxiliary strand that functions as an input binding region and is connected to a linker extending from one strand of the modular logic unit, either A strand or B strand). A NAND gate is FALSE, i.e., low fluorescence, when all input binding regions on the auxiliary strands bind to their analyte having a complementary nucleotide sequence.

An IMPLY gate is a DNA logic gate complex comprising at least one YES modular logic unit and at least one NOT modular logic unit parallelly integrated into a DNA board.

Herein, it is demonstrated that two YES modular logic units parallelly arranged on the DNA board can make a OR gate; two NOT modular logic units parallelly arranged on the DNA board a NAND gate; and a YES modular logic unit and a NOT modular logic unit parallelly arranged on the DNA board an IMPLY gate.

These DNA logic gates can be applied to real diagnosis of disease by dropping the logic gate complex solution to bio-samples such as blood, serum, urine and other liquid form of samples, or by coating a substrate with the logic gate complex to be used like a pH test strip. After letting the logic gate complex contact with the sample, a few minutes of waiting at room temperature (22° C.-25° C.) would display the fluorescence True/False result.

At least in a couple of aspects, these are novel designs. First, there are no or very few OR or NAND gates or no IMPLY gates that can recognize multiple inputs at the same time. Second, these gates reduce false signal by not employing displaced-strand releasing system. The DNA logic gates disclosed here are all designed to relay any input binding to the fluorescence probe at least indirectly, without released strand's diffusion and another toehold binding, which takes diffusion time and increases error rate.

By using these gates at the same time, the DNA logic gates provide another advantage. In a real disease situation, some miRNAs have up-expression while others have down-expression. The OR gate can be utilized to detect any increase of oncogenic miRNAs, and the NAND gate to detect any decrease of tumor suppressor miRNAs.

As an application example for the DNA logic gates, the proposed designs were tested for the detection of biomarker miRNAs for hepatocellular carcinoma (HCC) to evaluate and understand their performance. Early non-invasive detection can play a crucial role in the success of hepatocellular carcinoma (HCC) patient survival, which is the 3rd leading cause of cancer death.

Simultaneous detection of circulating microRNA(miRNA) that are abnormally up- or down-regulated can be the key to early HCC diagnostics leading to prompt treatment. The test results showed that the proposed designs can successfully function in terms of accuracy, efficiency, and speed. These logic units also show robust response when stored at ˜25° C. through a 3-month period.

Therefore, the DNA logic gate complexes disclosed herein can be used for the diagnosis of HCC, and the same method can be applied to diagnose other various diseases such as cardiovascular, neurodegenerative, immune diseases, in addition to cancer. In addition, it can be contemplated to use the DNA logic gates as biosensors for imaging.

In summary, the DNA logic gates disclosed here have the following advantages compared with prior art.

First, most prior art on the integration of DNA logic units into a DNA board uses DNA origami. This type of scaffold is a long single-stranded viral DNA folded by an excess of short oligonucleotides called staples, which limits the sequence directory during gate and architecture designing, and it is reported to have low assembly efficiency. The DNA logic gates disclosed here comprises a DNA board and at least one DNA modular logic gate, and they are composed of customizable synthetic oligonucleotides of 22-150 nt size, which allows (i) flexibility in sequence and architectural design, (ii) none to low undesired interactions of logic unit and scaffold strands, and (iii) high assembly efficiency.

Second, according to some reports, for the integration of logic gates into a DNA board, typically an excess amount of fuel strands (strands that are additionally added as intermediate operators to modulate the logic response, this fuel is different than input and output nucleic acids) are required. This invention does not require such intermediate fuel strands, which gives full autonomy to the DNA nanostructure to operate in cell environments without further human manipulation.

Third, some prior art reported local spacing between two or more logic units to be as small as 5 nm distance, which requires capping strands to avoid leaking interaction of the logic units in the lack of input. This invention distances each gate at 3.4 nm, which is the shortest reported distance between gates (to our knowledge) that does not require capping/blocking strands in the logic units. Therefore, this invention minimizes additional computing features required in other works.

In addition, no prior work has realized a universal gate NAND from only NOT logic units, thus our DNA logic units have a novel and unique integration design that allows simple and low-component NAND gate manufacturing.

The long-term goal here is to develop a molecular DNA automaton that can analyze the complex pattern of biomarkers followed by producing a single digital output:0 (for healthy) and 1—for patients requiring treatment. It can be envisioned to use each DNA logic unit simultaneously within a single test tube. Furthermore, the operability of this technology can be explored in vitro.

Overall, the invention is able to program different logic operators which in electronic computers is known as universality. The purpose of this invention is to design a customizable and programmable logic circuitry at low manufacturing cost, which has a potential to be scaled to DNA molecular computers.

To this end, in this disclosure, DNA logic gates are developed based on three Boolean logic circuits (OR, IMPLY, and NAND) for molecular diagnosis of hepatocellular carcinoma (HCC) by detecting microRNA biomarkers and molecular beacon fluorescence in order to indicate healthy or cancerous conditions.

### BRIEF DESCRIPTION OF THE DRAWINGS

FIG. 1. Design Philosophy

FIG. 2. Components for functionally complete 4J gates. (a) The 4J YES modular logic unit before (left) and after (right) input recognition, digital output 0 and 1, respectively. (b) The 4J NOT gate in the absence (left) and presence (right) of the input; digital output 1 and 0, respectively. Labels Q and F in the MB strands represent a molecular quencher and a fluorophore, correspondingly. (c) DNA board. The grey shaded area represents the ssDNA region within the DNA board accessible for hybridization with the gate units. The dashed lines represent the oligoethylene glycol spacers (see Table 1 for details). The duplexes between rail fragments and complementary fragments of Staple 1, Staple 2, or the gate units are 10-11 base pairs, which correspond to one helical turn in B-DNA.

FIG. 3. Functionally complete 4J gates integrated on DNA board. The 4J YES 1 (a) and NOT 2 gates (c) on the DNA board, both in the absence of input; ssDNA blocker strands (blck A1, A2, B1 and B2) fill the Rail fragments lacking the gates. Fluorescence response of 4J YES 1 (b) and 4J NOT 2 (d), respectively.

FIG. 4. DNA board properties. FIG. 4A. DNA scaffold/board expanding capacity. The scaffolding unit allows easy expansion to hold one or more logic units. FIG. 4B. DNA scaffold/board modular assembly capacity. Two or more DNA boards can be connected to increase the board size and circuit integration.

FIG. 5. Molecular circuitry and the truth table of OR, NAND, and IMPLY logic gates.

FIG. 6. Molecular circuitry and experimental results of OR, NAND, and IMPLY logic gates.

FIG. 7. Boolean logic symbols and truth tables. (a) Singleton YES (top) and NOT (bottom) gates. (b) connecting YES 1+NOT 2 to make IMPLY logic. (c) connecting two NOT modular logic units (NOT 2+NOT 3) to obtain a NAND logic function.

FIG. 8. YES 1+NOT 2=IMPLY. FIG. 8(a) Localization and connectivity of YES 1 and NOT 2 on the DNA board. FIG. 8(b) The 8% native PAGE-50 mM MgCl₂ results. L: dsDNA markers with their length, in base pairs, indicated to the left, 1: YES 1 gate strands (A1+B1), 2: NOT 2 gate strands (A2+B2+Bridge), 3: DNA board only, 4: IMPLY full assembly (YES 1+NOT 2+DNA board). The blue arrow indicates the fully assembled IMPLY gate nanostructure. FIG. 8(c) Fluorescence of IMPLY upon excitation at 555 nm. Red dashed line represents an experimental threshold, which was calculated as the average fluorescence of YES 1's output 0 plus three standard deviations. FIG. 8(d) Expected structural changes in the IMPLY nanostructure for the four Input 1/Input 2 combinations: digital inputs 0, 0; 1, 0; 0, 1; 1, 1.

FIG. 9. dPAGE analysis of full IMPLY assembly. The 12% dPAGE-8 M urea results. Lane 1: ssDNA markers with their lengths, in nucleotides, indicated; 2-10: individual ssDNA components of the IMPLY assembly; 11: DNA board; 12: IMPLY assembly before PAGE extraction. 13: IMPLY assembly after PAGE extraction. Blue arrowheads indicate the mobility of B2.

FIG. 10. Individual response of NOT 3 on DNA board. (a) The 4J NOT 3 gate on DNA board; left: in the absence of input; right: in the presence of input; ssDNA blocker strands blck A2, and B2 hybridized to ssDNA board area lacking gates. (b) The 4J NOT 3 fluorescence response after exciting at λ:555 nm.

FIG. 11. NOT 2+NOT 3=NAND. (a) Schematic representation of localization and connectivity of NOT 2 and NOT 3. (b) The 8% native PAGE-50 mM MgCl₂ results. L: dsDNA markers with their length, in base pairs, indicated, 1: NOT 3 gate strands (A3+B3), 2: NOT 2 gate strands (A2+B2+Bridge), 3: DNA board only, 4: NAND full assembly (NOT 2+NOT 3+DNA board). (c) Fluorescence response of NAND upon excitation at 555 nm. Red dashed line represents an experimental threshold, which was calculated as the average fluorescence of NAND's output 0 plus three standard deviations.

FIG. 12. dPAGE analysis of the full NAND assembly. The 12% dPAGE-8 M urea results. Lane 1: ssDNA markers with their lengths, in nucleotides, specified; 2-10: individual ssDNA components of the NAND assembly; 11: DNA board; 12: NAND assembly before PAGE extraction. 13: NAND assembly after PAGE extraction. Blue arrowheads indicate the mobility of B2.

FIG. 13. OR gate shelf-life. a. S/B=Output 1/Output 0. b. Native PAGE analysis.

FIG. 14. Constructing DNA logic gates with YES and/or NOT modular logic units; OR (A), IMPLY (B), and NAND (C) Boolean functions. (A) OR logic gate is achieved by connecting two DNA YES modular logic units integrated into the DNA board. The output sequence made by proximal cooperativity of the purple fragments (A1 and B1) gives a digital 1 when the YES 1 modular logic unit is turned ON either directly by Input 1 or indirectly by the output of YES 2, which follows the truth table. (B) IMPLY logic gate is realized by connecting YES 3 with NOT 4 modular logic units on the DNA board. The output sequence made out of the proximity of the green fragments of the (NOT 4 modular logic unit) A4 and B4 serves as another input for the YES 3 modular logic unit. Therefore, the IMPLY gate is OFF only when Input 4 is present alone, according to the truth table. (C) NAND logic gate is made by connecting two DNA NOT modular logic units (NOT 4 and NOT 5) on the DNA board. As NOT 5 exhibits high output in the absence of one or both inputs (Input 5 or 6), NAND logic gate would turn OFF only when both inputs are present, as expected from the truth table. Panels (A-C) show the connected YES and NOT logic symbols at the left. Black dots represent the connecting points of the logic gates for input uptake; gray lines represent the connectivity of the upstream and downstream gate that is turned on (turned black) in the absence of Input 1, Input 3, or Input 5.

FIG. 15. Setting up the materials and reagents. (A) Arrangement of 15 different microcentrifuge tubes (represented by circles) filled with solutions needed for constructing and testing the three DNA logic circuits: one tube with DNA grade water (black), one tube containing the assembled DNA board (brown), three tubes with different YES modular logic units (orange), two tubes with different NOT modular logic units (blue), two tubes with the MB probe solutions to report the output of the IMPLY/OR circuit and NAND circuit (purple), and six tubes containing individual inputs (green). (B) A tube-holder box made of black cardstock paper for holding samples containing four different input combinations of one DNA logic circuit. The samples are irradiated with a blue LED flashlight for output visualization at a 90° angle. (C) All necessary materials and reagents can be conveniently packaged into a 19×16×8 cm box as a portable education kit for the DNA molecular computing activity.

FIG. 16. Outline of the two-step experimental procedure for the construction of the DNA circuits and their testing with different input combinations. Use of a vortex/mixer and centrifuge is considered optional. Alternatively, samples can be mixed by gently flicking the tubes, and the solutions can be forced to the bottom of the tube by tapping the tube against a surface.

FIG. 17. Data interpretation. Top: visual fluorescence readout of OR, IMPLY, or NAND logic circuits depends on the input combination. The sequences of Input 1 correspond to hsa-miR-21-5p, Inputs 2 and 3 to hsa-miR-221-3p, Inputs 4 and 6 to hsa-miR-409-3p, and Input 5 is the DNA complement of hsa-miR-221-3p. Bottom: key definitions of digital values for inputs and outputs.

### DETAILED DESCRIPTION

### A. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “some embodiment,” “certain embodiment,” means that a particular feature, structure or characteristic described in connection with the embodiment is included in at least one embodiment of the present invention. Thus, appearances of the phrases “in one embodiment,” “in some embodiment,” or “certain embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The term “nucleotide” as used herein refers to a subunit of a nucleic acid (whether DNA or RNA or an analogue thereof) which may include, but is not limited to, a phosphate group, a 5-carbon sugar group (either ribose in RNA or deoxyribose in DNA), and a nitrogen-containing base, as well as analogs of such sub-units. Other functional groups (e.g., protecting groups) can be attached to the sugar group or nitrogen-containing base group. The bases in DNA are adenine (A), cytosine (C), guanine (G) and thymine (T) (in RNA, uracil (U)), but also modified purine and pyrimidine bases and other heterocyclic bases which have been modified (these moieties are sometimes referred to herein, collectively, as “purine and pyrimidine bases and analogs thereof”) can be considered. Such modifications include, e.g., diaminopurine and its derivatives, inosine and its derivatives, alkylated purines or pyrimidines, acylated purines or pyrimidines, thiolated purines or pyrimidines, and the like, or the addition of a protecting group such as acetyl, difluoroacetyl, trifluoroacetyl, isobutyryl, benzoyl, 9-fluorenylmethoxycarbonyl, phenoxyacetyl, dimethylformamidine, N,N-diphenyl carbamate, or the like. The purine or pyrimidine base may also be an analog of the foregoing; suitable analogs will be known to those skilled in the art and are described in pertinent texts and literature. Common analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl) uracil, 5-(methylaminomethyl) uracil, 5-(carboxymethylaminomethyl)-uracil, 2-thiouracil, 5-methyl-2-thiouracil, 5-(2-bromovinyl) uracil, uracil-5-oxyacetic acid, uracil-5-oxyacetic acid methyl ester, pseudouracil, 1-methylpseudouracil, queuosine, inosine, 1-methylinosine, hypoxanthine, xanthine, 2-aminopurine, 6-hydroxyaminopurine, 6-thiopurine, and 2,6-diaminopurine.

The term “oligonucleotide” or “nucleic acid” as used herein refers to any polyribonucleotide or polydeoxyribonucleotide that may be unmodified RNA or DNA or modified RNA or DNA. Thus, for instance, nucleic acids as used herein refers to, among others, single and double-stranded DNA, DNA that is a mixture of single and double-stranded regions, single and double-stranded RNA, and RNA that is mixture of single and double-stranded regions, hybrid molecules comprising DNA and RNA that may be single-stranded or, more typically, double-stranded or a mixture of single and double-stranded regions.

As used herein, the term “biomarker” refers to a biological marker that defines various types of objective indicators of health or diseases, i.e., medical signs observed from outside the patient or measurable substances whose presence or absence, or increased or decreased level in an organism is indicative of the presence or progress of disease, infection, or environmental exposure. Certain molecules, histologic staining patterns, radiographic or physiologic characteristics, which can be measured accurately and reproducibly, are examples of biomarkers. Biomarkers can also be used to monitor the efficiency of treatment, however biomarkers are not an assessment of symptoms.

As used herein, the term “microRNA (miRNA)” refers to a small single-stranded RNA (approx. 20-23 nucleotides). In eukaryotes, miRNAs play key roles in gene silencing. They are generated from initial transcripts (primary microRNA (pri-miRNA)) produced by RNA polymerase II (an enzyme for transcription of precursor mRNAs) or, in some cases, RNA polymerase III (an enzyme for transcription of 5S ribosomal RNA, tRNA and other small RNAs). Pri-miRNA folds into a hairpin structure, which has a terminal loop, a stem, and 5′- and 3′ terminal unpaired flanking sequences. Pri-miRNA undergoes strand cleavage process in the nucleus by the microprocessor complex which cuts 11 bp from the ssRNA-dsRNA junction on the ds RNA stem to remove the unpaired flanking sequences and form a precursor microRNA (pre-miRNA: stem and loop structure). The pre-miRNA is exported to the cytoplasm and cleaved to form a microRNA duplex (miRNA: miRNA* (passenger strand*)). The microRNA duplex unwinds to release a mature miRNA, which assembles to form a multiprotein complex, RNA-induced silencing complex (RISC) comprising Argonaute protein. When a miRNA on the RISC complex finds and base-pairs with a complementary sequence on the target mRNA, Argonaute protein cleaves the target mRNA through its intrinsic RNase activity, which is known as RNA interference (RNAi) or gene silencing. As a result, the target mRNA is degraded or its translation is somehow suppressed, depending on the degree of complementarity between the miRNA and the target mRNA target. (Macfarlane L A, Murphy P R. MicroRNA: Biogenesis, Function and Role in Cancer. Curr Genomics. 2010 November; 11(7):537-61.) Some miRNAs are biomarkers of neurologic disorders, sepsis, cardiovascular diseases or cancer.

As used herein, the term “logic gate” refers to a system performing a Boolean logic operation with one or more binary inputs to calculate a single binary output. The term “DNA logic gate” refers to an assembly of DNA strands that may comprise a non-DNA strand. A DNA logic ate performs a Boolean logic operation based on Watson-Crick base-pairing induced by physical or chemical inputs to generate a single output.

As used herein, the term “Boolean logic” refers to mathematical logic based on Boolean algebra. In contrast to elementary algebra, in which the values of the variables are numbers, in Boolean algebra, the values of the variables are true (1) and false (0). In addition, elementary algebra uses arithmetic operators such as addition, multiplication, subtraction, and division, whereas Boolean algebra uses logical operators such as conjunction (and: ∧), disjunction (or: ∨), and the negation (not: ¬). The term “universal gate” refers to a gate which can be implemented with any Boolean logic gate without using any other type of gates. The NAND (AND followed by NOT function) and NOR (OR followed by NOT function) are examples of universal gates. Boolean logic gates can accept and process the digital values of multiple inputs but produce only a single output. Each Boolean logic has a predetermined input(s), yielding a specific output set, defined by truth tables. For example, the truth table of OR logic gate dictates output is digital 1 when either or both inputs are digital 1; in NAND's logic gate, output is digital 0 only when both inputs are digital 1, and for IMPLY's logic gate, the output digital 0 is obtained only in a specific input value combination. In Boolean algebra, OR, IMPLY, and NAND logic are important to construct more complex computational circuits. A circuit is a set of logic gates purposely connected to achieve the desired output. For example, in electronic computers, the circuits are realized by connecting each logic gate and integrating them on boards made out of semiconductor materials, where the circuits direct the flow of electrons based on the programmed Boolean logic. The digital value of a circuit's output is dictated by the combination of values of multiple inputs, enabling output computing based on particular input combinations, a quality needed for personalized medicine.

The term “probe” as used herein refers to an oligonucleotide comprising a nucleic acid sequence of variable length for the detection of identical, similar, or complementary nucleic acid sequences by hybridization.

The term “molecular beacon” as used herein refers to a small stem-loop or hairpin structure of short DNA having a fluorophore moiety attached to one end and a quencher moiety attached to the other end, which is widely used for real-time detection of specific RNA/DNA sequences. The GC rich stem enables the quencher and fluorophore to remain in proximity for efficient quenching in the absence of a complementary sequence to the stem sequence. Upon hybridization to a complementary sequence, an MB probe opens into an elongated conformation, and fluorescence can be detected. For multiple types of DNA logic gates, different fluorophores can be conjugated to MBs for each type of the gate, and different quenchers according to the fluorophores. In the invention disclose here, molecular beacon probes are for output readout. In these DNA molecular computing designs, inputs and outputs correspond to nucleic acid sequences. The output is a new nucleic acid sequence generated after computing all inputs, and it can be detected using a complementary DNA oligonucleotide tagged with a fluorophore and a quencher at its opposite termini, known as a molecular beacon (MB) probe. In the absence of a complementary output, MB is in a hairpin conformation, which keeps the fluorophore near the quencher, ensuring a low fluorescence. Upon the binding of MB to its complement, it stretches and distances the fluorophore from the quencher, enhancing the fluorescence signal. Therefore, MB helps in transducing the nucleic acid output to a fluorescence signal and easily monitoring the molecular computing readout. In this laboratory experiment, high fluorescence intensity was interpreted as digital output 1 and low fluorescence as digital output 0.

The term “hybridization” as used herein refers to the process of association of two nucleic acid strands to form an anti-parallel duplex stabilized by means of hydrogen bonding between residues of the opposite nucleic acid strands. The terms “hybridizing” and “binding”, with respect to polynucleotides, are used interchangeably and is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequence has a T, G, C or A, respectively. The hybridized fragment/oligonucleotide is called a “duplex.” The term “hybridize to” as used herein refers to the binding and duplexing a nucleic acid molecule preferentially to a particular nucleotide sequence under stringent conditions.

The term “complementary” as used herein refers to a sufficient number in the oligonucleotide of complementary base pairs in its sequence to interact specifically (hybridize) with the target nucleic acid sequence to be amplified or detected. As known to those skilled in the art, a very high degree of complementarity is needed for specificity and sensitivity involving hybridization, although it need not be 100%.

The term “analyte” as used herein refers to an oligonucleotide or polynucleotide for which it is desired to detect. The analyte for use in the methods herein disclosed may be an oligonucleotide or a polynucleotide, immobilized on a solid support or in free solution, which is isolated from a plant or animal host, a cultured cell or a cell or population of cells in a tissue of a plant or animal. Examples of suitable analytes for the invention comprise mRNA, long-noncoding RNA (lncRNA), microRNA (miRNA), small interfering RNA (siRNA), small nucleolar RNA (snoRNAs), Piwi-interacting RNA (piRNA), tRNA-derived small RNA (tsRNA) and rRNA-derived small RNA (srRNA), and the like.

### B. Overview

### 1. Molecular Computing by DNA Logic Gates

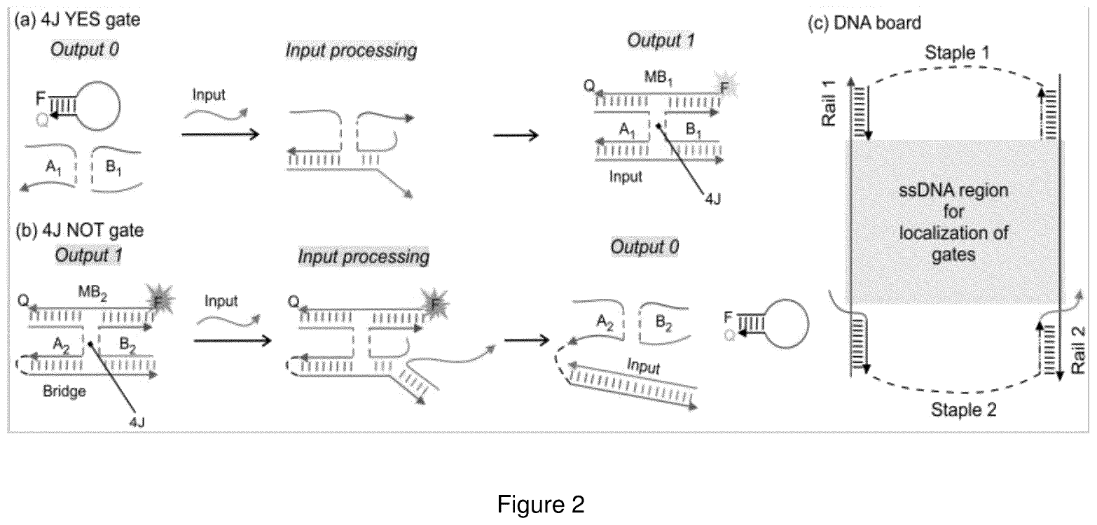

Building DNA logic gates and circuits is possible due to the predictability of Watson-Crick base-pairing, where adenosine (A) pairs with thymidine (T), and guanosine (G) pairs with cytidine (C). This predictable base-pairing is used to program Boolean logic functions that operate through the association and dissociation of DNA fragments. Modulating sequences of DNA molecules as DNA logic gates and circuits can yield higher-ordered DNA structures not found in nature (e.g., DNA board in FIG. 2C). This laboratory activity introduces DNA as a building material of molecular computer hardware, a function different from the natural role of DNA as storage of genetic information.

In DNA molecular computing, the development of individual logic gates is still in its infancy. Here are shown the integration of DNA modular logic unit gates into circuits by their localization onto a DNA board, an approach that mimics Si-based circuits. The DNA board is a molecular structure assembled in aqueous solution, where it provides spatial localization of two modular logic units (YES and NOT) for their integration into DNA circuits.

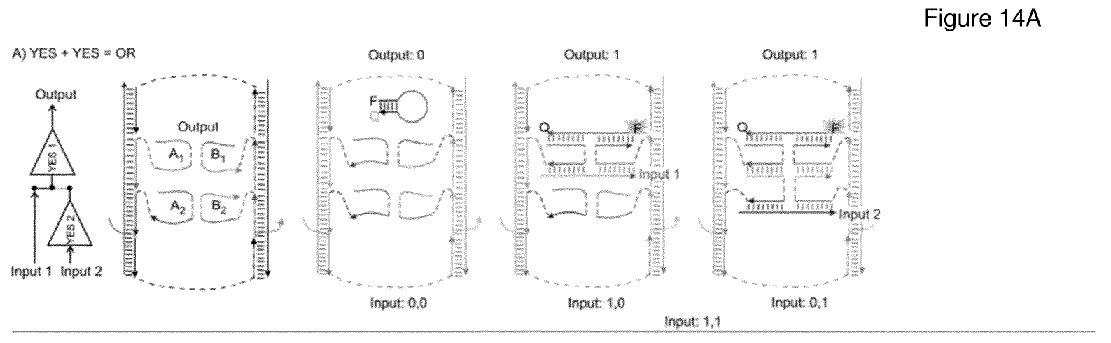

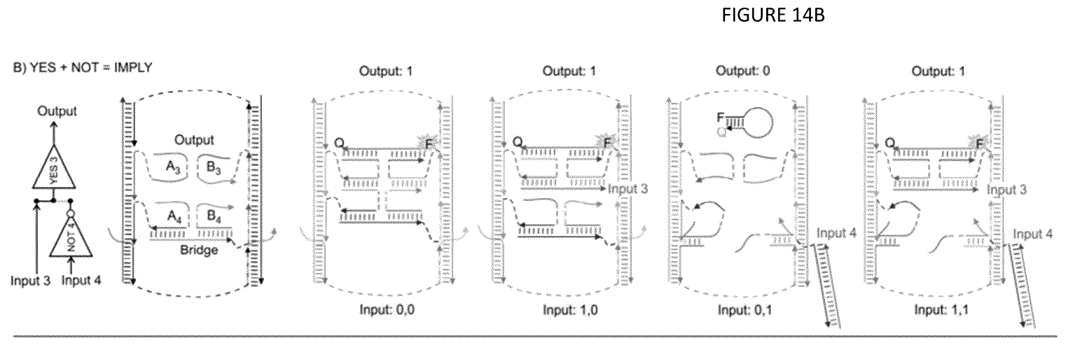

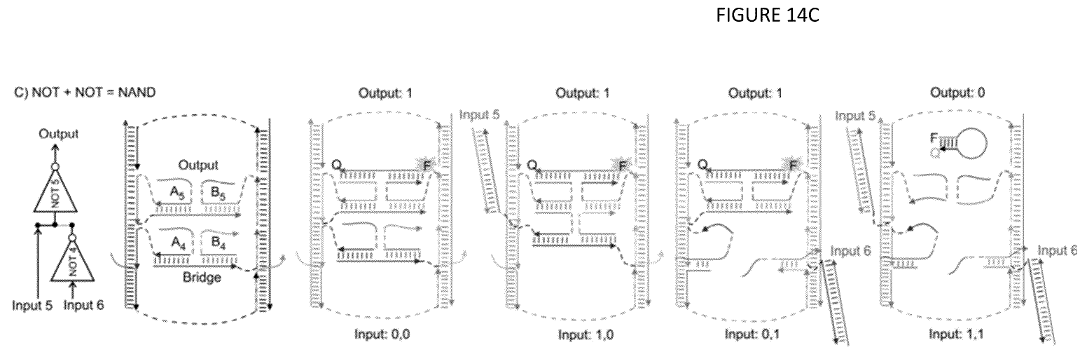

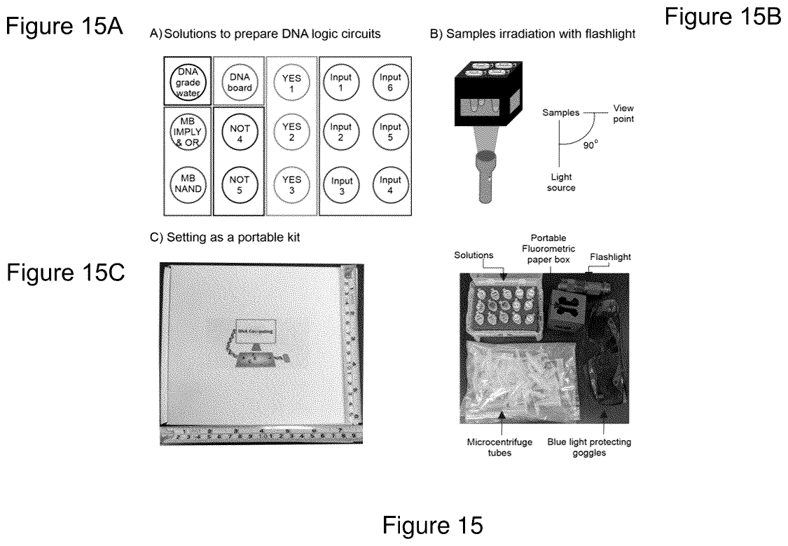

The DNA YES and NOT modular logic units preserve their Boolean truth table by binding to only one input at a time, either an input externally added to the DNA circuit or an input relayed from the downstream gate (FIG. 14). In Boolean algebra, achieving OR, IMPLY, and NAND logic by connecting YES and NOT modular logic units has not been reported. However, computations made out of DNA can follow different connectivity paradigms.

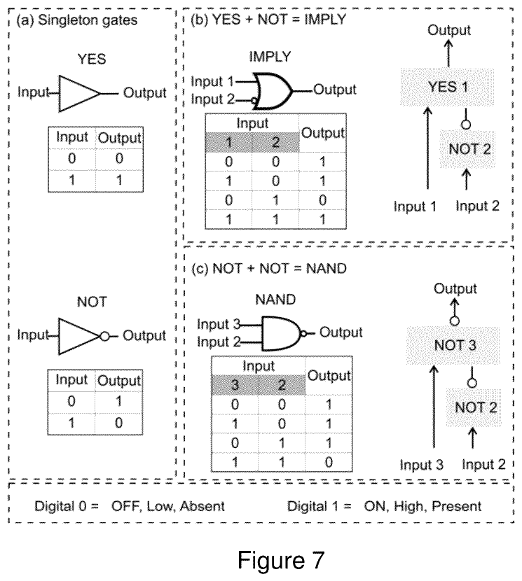

Boolean logic gates are the most basic components in electronic computers. [1] A set of AND, OR, and NOT gates is a well-known functionally complete set in digital computers. [1] This set has attracted attention because of its universality—the ability to achieve any other logic functions by integrating multiple units of this limited set. [2] This modular and scalable approach enables easy design and cost-efficient manufacturing of computational circuits. IMPLY and NAND are ‘universal’ (or functionally complete) gates, each of them sufficient to build semiconductor circuits of arbitrary complexity. [3] The IMPLY logic produces a low output only when the conditional set (Input 1: low, and Input 2: high) is true (FIG. 7b). Lately, IMPLY has also attracted attention for its use in ‘memristive’ switches, memory resistors that perform logic operations.4,5 The simplest Boolean logic gates are YES and NOT: YES produces a high output in the presence of the input, and a low output in its absence (FIG. 7a top). NOT is the inverter of a YES logic (FIG. 7a, bottom). In digital computing, neither the combination of YES and NOT gates, nor NOT gate alone, have never been reported to comprise a functionally complete set of gates.

A DNA logic gates connected to each other via DNA four-way junction (4J) structures are developed here. [11,12] The gates recognize nucleic acid sequences as inputs and induce an arrangement of a new sequence by bringing in proximity two oligonucleotide fragments. The new output binding sequence can be conveniently detected by a molecular beacon (MB) probe- a fluorophore- and a quencher-labelled DNA hairpin.[13] The change in fluorescence from the opening/closing of the MB probe can be correlated to the binary response (0 and 1) as in digital computing.

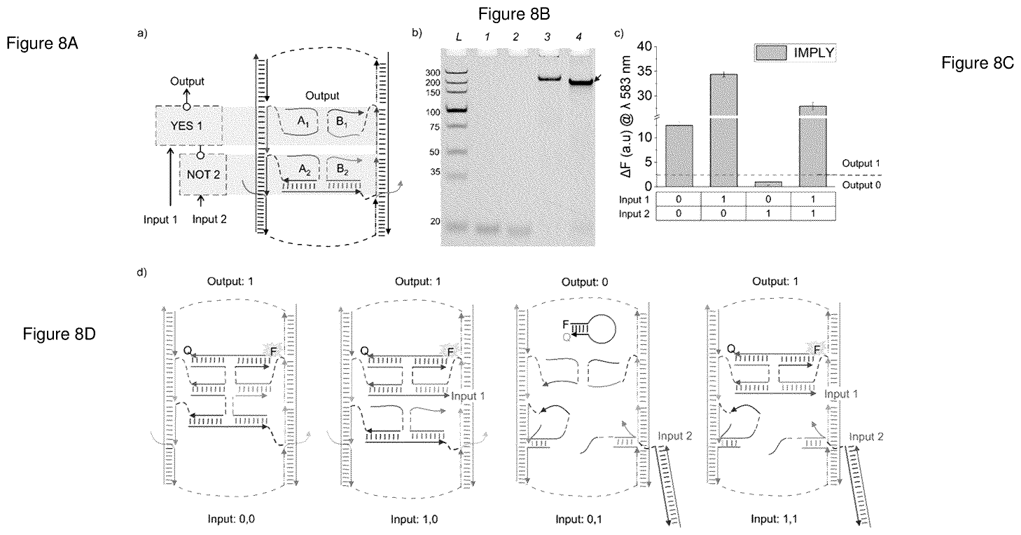

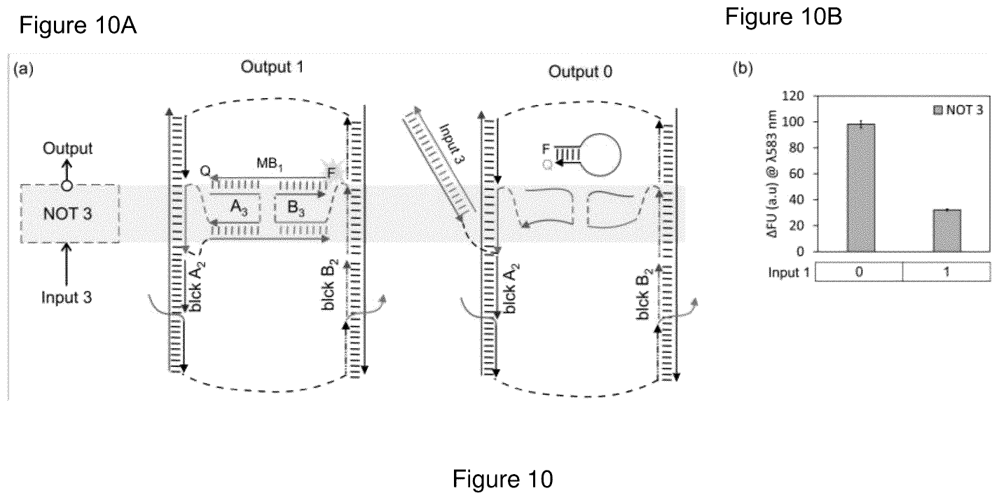

FIG. 8a illustrates a 4J YES Boolean logic gate with the output sequence (A1+B1) triggering the MB₁ probe opening after input recognition. In the 4J NOT gate (FIG. 8b), strands A2 and B2 are brought together by a DNA “bridge”, which stabilizes their hybridization with the MB₂ probe in the absence of the input, thus enabling a high fluorescent signal (digital 1). When an oligonucleotide input hybridizes to the Bridge of a 4J NOT gate, it displaces A2 and B2 resulting in the dissociation of the MB₂ probe to exhibit low fluorescence (digital 0) (FIGS. 8b, 10). Here, it is reported that a set of connected YES and NOT, or two NOT gates can lead to functionally complete IMPLY or NAND gates, respectively.

In summary, a functionally complete Boolean operator is sufficient for computational circuits of arbitrary complexity. We connected YES (buffer) with NOT (inverter), and two NOT four-way Junction (4J) DNA gates to obtain IMPLY and NAND Boolean functions, respectively, each of which represents a functionally complete gate. The results show a technological path towards creating a DNA computational circuit of arbitrary complexity based on singleton NOT or a combination of NOT and YES modular logic units, which is not possible in electronic computers. Therefore, DNA-based circuits and molecular computation may offer opportunities unforeseen in electronics.

One common paradigm in developing a molecular computer follows the path established by semiconductor computer technology. This includes designing a functionally complete sets of Boolean logic gates, connecting the logic gates in circuits by integrating modular logic units into a common platform, powering using (bio)chemical reactions, and achieving an easily readable signal for convenient communication with a human operator [12,14]. Applications of such computational systems in controlling gene expression and in diagnosing infectious diseases and cancer have been envisioned[15,16,17,18,19]. Thus, computers made of molecules can be explored for the application of well-developed computational living systems.

This disclosure demonstrates that molecular (DNA) computational systems may offer opportunities unrealized in electronics. Indeed, an electronic set of YES and NOT modular logic units has never been considered as a complete set of Boolean gates. In this work, it is demonstrated for the first time that two YES modular logic units, two NOT modular logic units, and alternative YES and NOT modular logic units made of DNA, can be connected in a circuit that fulfils functionally complete gates, OR, NAND and IMPLY. This is possible because the YES modular logic units in the OR and IMPLY gates and NOT modular logic units in the NAND (and IMPLY) gate(s) recognize either the oligonucleotide input or the outputs of the upstream gates; the coexistence of these two distinct functions is a feature that is absent in the majority of other devices that fulfil the function of Boolean logic gates. Since all gates here, OR, NAND and IMPLY function sufficiently to make a circuit of arbitrary complexity, it is concluded that singleton {YES} and {NOT} and a doubleton {YES; NOT} gate can act as functionally complete sets in DNA-integrated computational circuits.

In conclusion, two YES modular logic units can make an OR function, and two NOT modular logic units can make a NAND function while two DNA 4J gates with YES and NOT Boolean functions can be connected to make an IMPLY gate. Theoretically, a computational circuit of any complexity can be built only from this set of DNA logic gates. This opens a route to building computational circuits of arbitrary complexity from simple YES and NOT DNA logic gates. This modular connectivity could ease the burden of developing new architectures when realizing new Boolean circuitries. Therefore, while developing molecular logic gates, we should look for opportunities that are unexpected from our experience with electronic computers.

The objectives of this invention are (i) to detect and interact with DNA and/or RNA analyte molecules, (ii) to execute an output after simultaneously identifying multiple DNA and/or RNA analyte molecules, and (iii) to enable modular integration to construct a variety of molecular circuits. That is, the DNA molecular circuits of this disclosure can identify multiple DNA and RNA sequences as well as their expression levels involved in genetic and pathogenic disorders. The invention can also be applied to multifarious analytes for a unanimous diagnosis.

The DNA computing nanostructure disclosed here is composed of several oligonucleotides, which are assembled to form two functionally distinct components of the DNA computing nanostructure (i) a DNA board composed of a pair of rail strands and a pair of staple strands and (ii) at least two DNA modular logic unit comprising a pair of nucleotide strands (arbitrarily, A strand and B strand).

As an input analyte, any DNA and/or RNA oligo- or polynucleotides can be contemplated. The nucleotide sequences of input binding regions in the YES and NOT modular logic units comprise complementary nucleotide sequences to the nucleotide sequences of target analyte nucleotides.

2. Molecular Diagnosis of Hepatocellular Carcinoma (HCC) by microRNA Detection

As an example for DNA molecular computing as a biomedical tool, the DNA circuits were designed to recognize as inputs microRNAs (miRs) correlated with the diagnosis of hepatocellular carcinoma (HCC), one of the most common types of liver cancer. miRs are noncoding RNA of a short length (18-25 nucleotides), lacking secondary structures in their mature form. miRs have been found to regulate processes like RNA gene expression/silencing and as signaling molecules of intercell communication. In their mature form, they can be found either in the cellular matrix or circulating in plasma. These features make them attractive candidates as biomarkers in the diagnosis of diseases. In cancer, more than one miR can be abnormally overexpressed and/or underexpressed. The precision of the diagnosis can be improved by considering as many miRs as possible. However, analyzing multiple miRs (each with different aberrant expression levels) can be a challenging task. We selected miRs whose over- and under-expression have been correlated with the development, tumor growth, and metastasis of HCC.

An OR logic gate was designed to recognize the selected overexpressed miR, while the NAND logic gate could identify an underexpressed miR. The IMPLY logic gate could bind to one overexpressed and one underexpressed miR. For experiments, the biological background of miRs is introduced, and how digital values could be assigned to their under- and overexpression during testing with each DNA logic circuit. To simplify interpretation of the DNA circuits' response, the sample was classified as “cancerous” if the miR input(s) triggered a high fluorescence signal. Conversely, low fluorescence indicated a “healthy” state.

### 3. DNA Board

The DNA board serves as a flexible hybridization scaffold for integrating multiple modular logic units. This DNA board is a novel design whose size and sequence can be customized without reaching chemical synthesis limitations.

The DNA board is composed of four strands-two rails and two staples for building a DNA circuit (FIG. 2c). The rail strand is a single stranded DNA, optionally with a length of at least 40 nucleotides, optionally 40-200 nucleotides; and the staple strand comprises a single stranded DNA. In one example, the staple strands are comprised of a spacer, such as oligoethylene glycol polymer, with a single stranded DNA connected at each end of the spacer. The staple strand can also be a single stranded DNA, whose length can be 30-240 n.t. (10-20 n.t. staple/rail hybridization+10-200 n.t. input/output binding region length+10-20 n.t. staple/rail hybridization).

To form a circuit, one ssDNA end of a first staple strand hybridizes to the 5′-end of a first rail strand and the other ssDNA end to the 3′-end of a second rail strand; and one ssDNA end of a second staple strand hybridizes to the 3′-end of the first rail strand and the other ssDNA end to the 5′-end of the second rail strand. The rail strand between the rail-staple hybridization regions is single stranded; and the single stranded region in the rail strand has at least two nucleotide sequences complementary to other DNAs for accommodating modular logic units (10-15 n.t. length per modular logic unit).

The modular logic unit here is a YES gate composed of A and B strands and/or a NOT modular logic unit composed of A and B strands or A, B′, and C strands, which are described below. For parallel arrangement of multiple modular logic units, the nucleotide sequences on the rail strands for partial hybridization to each of the A and B (or A and C) strands of a modular logic unit, are facing each other on the opposite sides of the DNA rail strands. When multiple modular logic units are arranged in the DNA board, there is no nucleotide gap on the rail strand between one modular logic unit binding sequence and the next modular logic unit binding sequence.

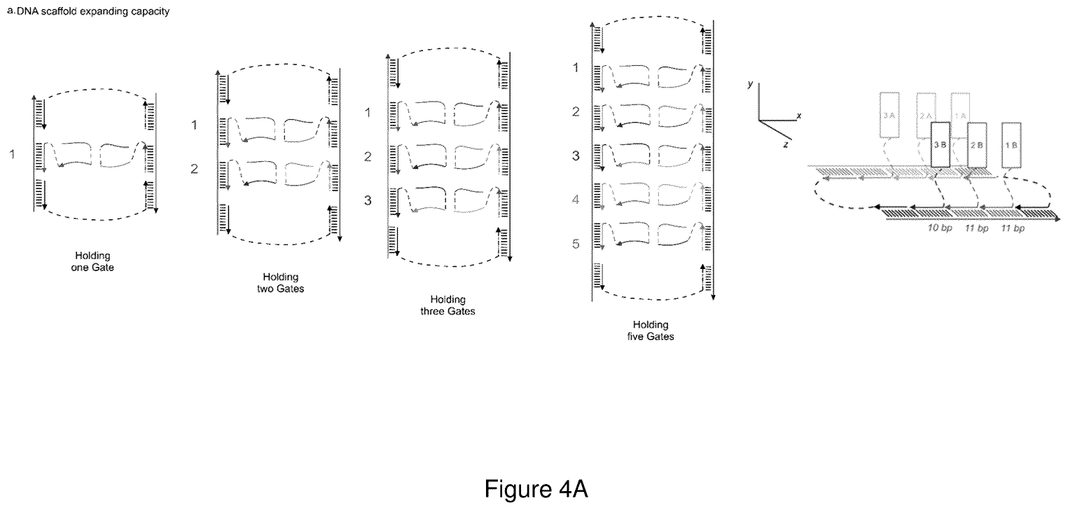

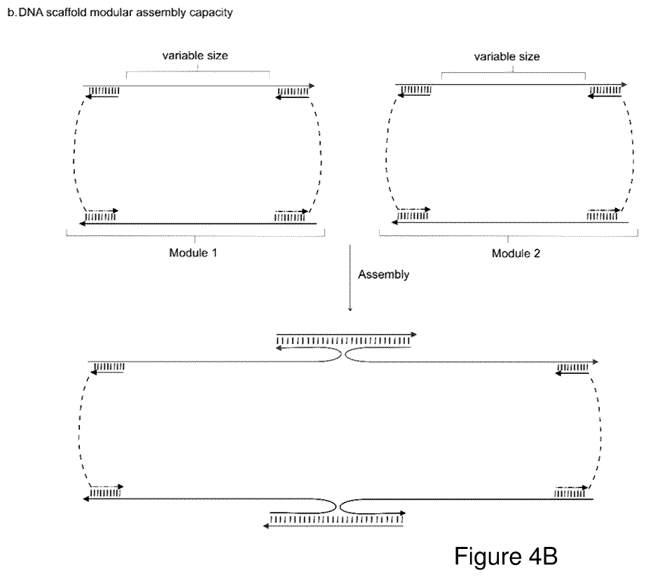

Additionally, homologous DNA boards can be connected using a pair of single stranded nucleotide fragments to increase the circuit size (FIG. 4). That is, two boards can be connected to form one board in the presence of a pair of single stranded oligonucleotide fragments (20-40 nucleotides) through denaturation and renaturation steps, wherein a first oligonucleotide fragment hybridizes to the 3′-end of a first rail strand of a first board (10-20 nucleotides) and to the 5′-end of a first rail strand of a second board (10-20 nucleotides) with or without a nucleotide gap between both hybridization regions on the fragment; and a second oligonucleotide fragment hybridizes to the 5′-end of a second rail strand of the first board (10-20 nucleotides) and to the 3′-end of a second rail strand of the second board (10-20 nucleotides) with or without a nucleotide gap between both hybridization regions on the fragment. In addition, more than two boards can be connected to form one board in the presence of more than a pair of single stranded oligonucleotide fragments in this manner.

### 4. YES Modular Logic Unit

A YES modular logic unit comprises a pair of single strands, arbitrarily A strand and B strand.
a. The A strand comprises, from 5′ to 3′ end direction, one half of an output binding region (e.g. 5-100 nucleotides), a first linker (e.g., an oligoethylene glycol spacer, optionally 18 oligoethylene glycol polymer of 10-20 nucleotide length), one half of an input binding region (e.g., 5-100 nucleotides), a second linker (e.g., an oligoethylene glycol spacer, optionally 18 oligoethylene glycol polymer of 10-20 nucleotide length), and a board hybridization region (e.g., 10-15 nucleotides).
b. The B strand comprises, from 3′ to 5′ end direction, the other half of an output binding region (e.g., 5-100 nucleotides), a first linker (e.g., an oligoethylene glycol spacer, optionally 18 oligoethylene glycol polymer of 10-20 nucleotide length), the other half of an input binding region (e.g., 5-100 nucleotides), a second linker (an oligoethylene glycol spacer, optionally 18 oligoethylene glycol polymer of 10-20 nucleotide length), and a board hybridization region (e.g., 10-15 nucleotides). Although the liker here is a non-nucleic acid linker, e.g., polyethylene glycol linker, it can be composed of or comprise a proper length of nucleotides.

The board hybridization region of the A strand hybridizes to a complementary nucleotide sequence in the single-stranded region of one rail strand (e.g., 10-15 nucleotides per modular logic unit); and the board hybridization region of the B strand hybridizes to a complementary nucleotide sequence in the single-stranded region of the other rail strand (e.g., 10-15 nucleotides per modular logic unit), so as that the DNA board serves as a flexible hybridization board for integrating the modular logic unit(s).

In the absence of an analyte, any of the input binding regions is not occupied, and both A and B strands of the modular logic unit are not fixed enough to expose all the nucleotides in the output binding region, and thus there is no MB binding nor MB fluorescence (input 0→output 0). However, in the presence of any input (analyte), A and B strands are stabilized enough to expose all the nucleotides in the output binding region so as to induce an MB binding to the output binding region. In this state, the first modular logic unit forms a stable 4-way junction with the input/input binding region double strand and the MB/output binding region double strand to generate fluorescence signal from the molecular beacon (input 1→output 1). (FIG. 2a). This 4WJ DNA structure can serve as a motif for the construction of elemental YES modular logic unit.

### 5. NOT Modular Logic Unit

A NOT modular logic unit can be composed of two strands (A strand and B strand that has a bridge strand comprising an auxiliary strand and a linker that extends from one end of the board hybridization region) or three strands (A strand, B′ strand that does not have a board hybridization region, and C strand having a board hybridization region and a bridge strand). Unlike the YES modular logic unit, in a NOT modular logic unit, the input binding region (complementary nucleotide sequence to the input/analyte) is on the auxiliary strand. The auxiliary strand is a single stranded DNA optionally with a length of 10-200 nucleotides, which comprises a nucleotide sequence complementary to an input (analyte);

In a NOT modular logic unit comprising a set of two single strands, A strand and B strand:
a. the A strand comprises, from 5′ to 3′ end direction, one half of an output binding region (e.g., 5-100 nucleotides), a first linker (e.g., an oligoethylene glycol spacer, optionally 18 oligoethylene glycol polymer of 10-20 nucleotide length), one half of an auxiliary strand binding region (e.g., 5-100 nucleotides), a second linker (e.g., an oligoethylene glycol spacer, optionally 18 oligoethylene glycol polymer of 10-20 nucleotide length), and a board hybridization region (e.g., 10-15 nucleotides);
b. the B strand comprises, from 3′ to 5′ end direction, the other half of an output binding region (e.g., 5-100 nucleotides), a first linker (e.g., an oligoethylene glycol spacer, optionally 18 oligoethylene glycol polymer of 10-20 nucleotide length), the other half of an auxiliary strand binding region (e.g., 5-100 nucleotides), a second linker (e.g., an oligoethylene glycol spacer, optionally 18 oligoethylene glycol polymer of 10-20 nucleotide length), a board hybridization region (e.g., 10-15 nucleotides), and a bridge strand comprising a third linker (e.g., an oligoethylene glycol spacer, optionally 9 oligoethylene glycol polymer of 5-10 nucleotide length) and an auxiliary strand.

In a NOT modular logic unit comprising a set of three single strands, A strand, B′ strand, and C strand:
a. the A strand comprises, from 5′ to 3′ end direction, one half of an output binding region (e.g., 5-100 nucleotides), a first linker (e.g., an oligoethylene glycol spacer, optionally 18 oligoethylene glycol polymer of 10-20 nucleotide length), one half of an auxiliary strand binding region (e.g., 5-100 nucleotides), a second linker (e.g., an oligoethylene glycol spacer, optionally 18 oligoethylene glycol polymer of 10-20 nucleotide length), and a board hybridization region (e.g., 10-15 nucleotides);
b. the B′ strand comprises, from 3′ to 5′ end direction, the other half of an output binding region (e.g., 5-100 nucleotides), a first linker (an oligoethylene glycol spacer, optionally 18 oligoethylene glycol polymer of 10-20 nucleotide length), the other half of an auxiliary strand binding region (e.g., 5-100 nucleotides); and
c. the C strand comprises from 5′ to 3′ end direction, a bridge strand comprising an auxiliary strand and a third linker (e.g., an oligoethylene glycol spacer, optionally 9 oligoethylene glycol polymer of 5-10 nucleotide length), and a board hybridization region (e.g., 10-15 nucleotides).

As in an YES modular logic unit, the board hybridization region of the A strand hybridizes to a complementary nucleotide sequence in the single-stranded region of one rail strand (e.g., 10-15 nucleotides per modular logic unit); and the board hybridization region of the B strand or C strand hybridizes to a complementary nucleotide sequence in the single-stranded region of the other rail strand (e.g., 10-15 nucleotides per modular logic unit), so as that the DNA board serves as a flexible hybridization board for integrating the modular logic unit(s).

In a NOT modular logic unit, in the absence of an analyte, the region corresponding to the input binding region of a YES modular logic unit is occupied by an auxiliary strand, giving structural stability to A and B strands of the modular logic unit, and the output binding region is stabilized enough to bind to an MB. In this state, a modular logic unit forms a stable 4-way junction with the auxiliary strand/auxiliary strand binding region double strand and the MB/output binding region double strand to generate fluorescence signal from the molecular beacon (input 0→output 1). However, in the presence of an input, the input binds to the auxiliary strand to peel it off from the auxiliary strand binding region, i.e., strand displacement, and leaves the region unoccupied. This change disturbs the whole gate structure, and the MB is released (input 1→output 0) (FIG. 2b).

### 6. OR Logic Gate

An OR logic gate is a parallel arrangement of multiple YES modular logic units, and the output that hybridizes to the output binding region of a first modular logic unit is a molecular beacon. If more than one modular logic unit is integrated into the DNA board, the output that hybridizes to the output binding region of a modular logic unit other than the first modular logic unit is an input binding region of a preceding modular logic unit, which is designed to be complementary to the output binding region of a subsequent modular logic unit. This is a novel design for communication with the preceding modular logic unit and eventually with the first modular logic unit. In the presence of a molecular beacon and an input (analyte) complementary to the nucleotide sequence of any input binding regions, the first modular logic unit forms a 4-way junction to generate fluorescence signal from the molecular beacon.

One aspect of an OR logic gate is that this gate generates fluorescence signal with any analyte binding to any of the YES modular logic units, and this can be utilized for the detection of biomarker nucleotides such as miRNA or siRNA when the biomarker nucleotides tend to increase in disease/disorder/condition. For example, when a set of biomarker miRNAs increase during a specific cancer, e.g., pro-cancer miRNAs, regardless of the individual miRNAs of the set, the total number of the miRNAs increases, and in a testing solution comprising relatively excessive amount of the OR logic gate complex, more YES modular logic units will be occupied by the miRNAs and True compared with non-cancer control sample.

### 7. NAND Logic Gate

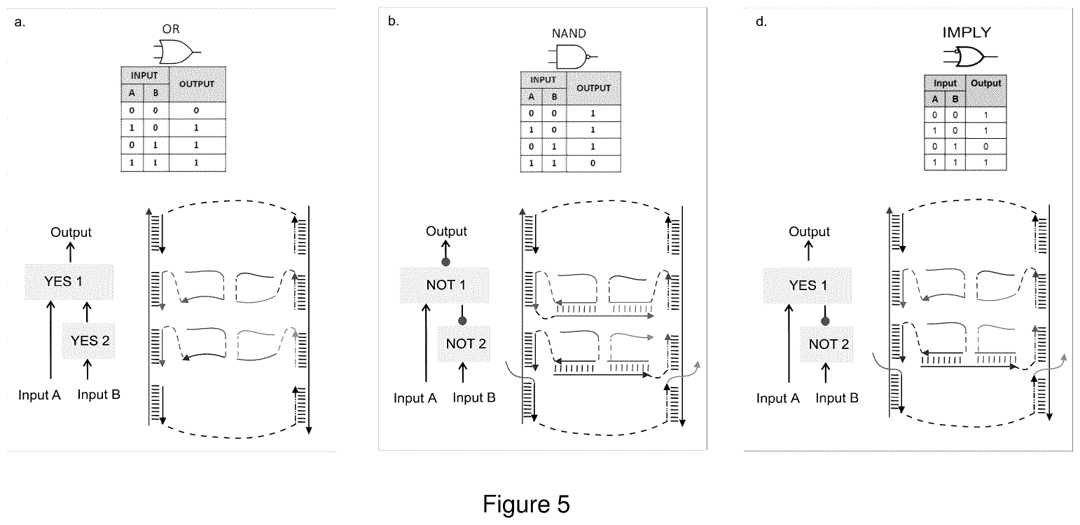

A NAND logic gate is a parallel arrangement of multiple NOT modular logic units. Regarding the arrangement of multiple NOT modular logic units having bridge strands into a DNA board, optionally, a NOT modular logic unit having a bridge strand extending from the 3′ end of the board hybridization region of A strand and a NOT modular logic unit having a bridge strand extending from the 5′ end of the board hybridization region of B strand may alternate with each other (FIG. 5).

As in an OR logic gate, in a NAND logic gate, the output that hybridizes to the output binding region of a first modular logic unit is a molecular beacon. However, in the NAND gate, when more than one modular logic unit is integrated into the DNA board, the output that hybridizes to the output binding region of a modular logic unit other than the first modular logic unit is an auxiliary strand binding region of a preceding modular logic unit, which is designed to be complementary to the output binding region of a subsequent modular logic unit. In the absence of an input, the auxiliary strand binding region of a modular logic unit is occupied by the auxiliary strand.

In the absence of all inputs, all auxiliary strands bind to the auxiliary strand binding regions of NOT modular logic units, and the whole gate structure is stable for MB binding (output 1). In the absence of some inputs, i.e., in the presence of some inputs, but not all inputs, some of the auxiliary binding regions are still occupied by some auxiliary strands, and the structural stability is transferred to the first modular logic unit through the output binding region/auxiliary strand binding region hybridization between neighboring modules. In this state, the 4WJ of the first NOT gate is stable and generates fluorescence (output 1).

However, in the presence of all inputs, i.e., all auxiliary strands hybridize to their complementary inputs instead of their auxiliary binding regions of the A and B (or B′) strands of the gates, all auxiliary strands are peeled off from their binding regions, which disturbs the whole gate structure, including the first NOT modular logic unit. In such a state when all NOT modular logic units are open, the hybridization between the output binding region and the unoccupied auxiliary binding region between neighboring modular logic units may not generate enough tension to keep the structural stability for MB binding to the first modular logic unit, and thus MB is released (output 0). That is, in the presence of a molecular beacon, only when all inputs (analytes) complementary to the nucleotide sequences of all input binding regions on the auxiliary strands are present, the first modular logic unit fails to form a 4-way junction and the fluorescence signal from the molecular beacon is quenched due to its hairpin structure.

One aspect of a NAND logic gate is that this gate turns off fluorescence signal with all analyte binding to all modular logic units, i.e., that the probability of True (in this case, less input region binding) increases with a decrease in a total number of analytes; and this can be utilized for the detection of biomarker nucleotides such as miRNA or siRNA when the biomarker nucleotides tend to decrease in disease/disorder/condition. For example, when a set of biomarker miRNAs decrease during a specific cancer, e.g., anti-cancer miRNAs, the probability of the presence of the whole set of miRNAs and consequently the probability of complete occupation of the input binding regions on the auxiliary strands decreases, which means an increase of fluorescence signal. That is, in a testing solution comprising relatively small ratio of the NAND logic gate complex to the pool of miRNAs in the sample, with a decreased number of anti-cancer miRNA, a smaller number of auxiliary strands will be peeled off from the NOT modular logic unit, and more NOT modular logic unit will be True in the cancer sample compared with non-cancer control sample having more biomarker analyte miRNAs.

### 8. IMPLY Logic Gate

An IMPLY logic gate is a combination of at least one YES modular logic unit and at least one NOT modular logic unit, which alternates with each other. In the EXAMPLES, a YES modular logic unit is the first modular logic unit and a NOT modular logic unit is the second, but a NOT modular logic unit can be the first modular logic unit and a YES modular logic unit can be the second.

In the presence of an input analyte (e.g., oncogenic miRNA) binding to a YES modular logic unit regardless of an input analyte (e.g., anticancer miRNA) binding to a NOT modular logic unit, an MB binds to the output binding region (output 1) of the first modular logic unit. In the absence of both input analytes for the Yes modular logic unit and for the NOT modular logic unit, the whole gate is still stabilized by the binding of an auxiliary strand to a NOT modular logic unit, and MB binding still occurs at the first modular logic unit (output 1) through neighboring input binding region of a YES modular logic unit and output binding region of a NOT modular logic unit. Even if the position of a YES modular logic unit and a NOT modular logic unit is reversed, the outcome is the same. However, in the absence of an input analyte for the YES modular logic unit and in the presence of input analyte for the NOT modular logic unit, the auxiliary strand of the NOT modular logic unit is peeled off from the gate to hybridizes to the input, and there is no strand to fix A and B strands at their positions, and this destabilizes the whole gate structure, so the MB is released (output 0).

### C. Embodiment Example

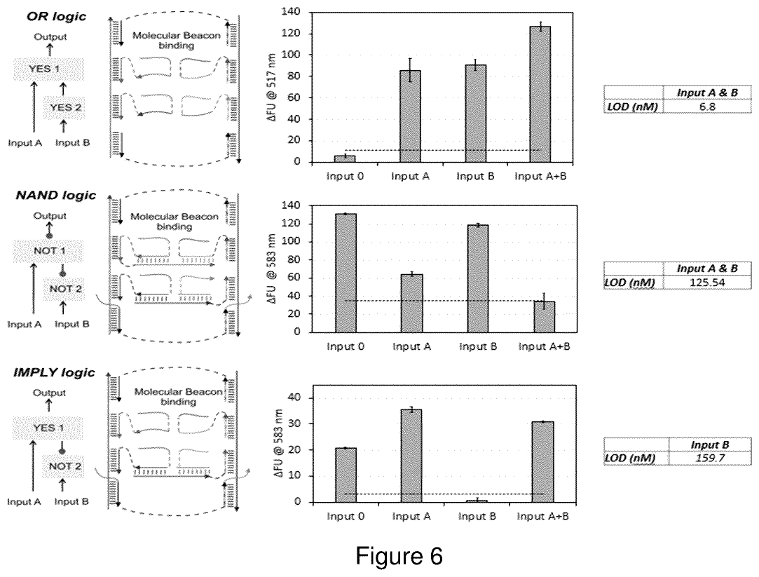

To show that these YES and NOT modular logic units have potential as building blocks for the construction of DNA logic gate circuits with sensing capabilities, the integration of two modular logic units were designed to build them up to a two-layer DNA logic gates; OR (YES-YES), NAND (NOT-NOT), and IMPLY (YES-NOT) gates (FIGS. 5 and 6).

Each of these two-layer logic gates in FIGS. 5 and 6 aims to respond as TRUE or FALSE to the question. As an example, a question was asked: are these miRNA expressions cancerous? For instance, OR logic gate gives TRUE in the presence of either or both oncogenic miRNAs, NAND logic gate gives TRUE in the absence of both—or either tumor suppressor miRNAs, but gives FALSE in the presence of both tumor suppressor miRNAs. In addition, IMPLY logic gate gives TRUE as long as one oncogenic miRNA (input 1) is present regardless of the presence or absence of a tumor suppressor miRNA; and in the absence of input 1 (no oncogenic miRNA), the absence of tumor suppressor miRNA (input 2) gives TRUE. However, the absence of oncogenic miRNA (input 1) and the presence of a tumor suppressor miRNA (input 2) gives FALSE.

To investigate the potential of this technology, five miRNAs associated with hepatocellular carcinoma (HCC) were selected as inputs and analyzed their performance in solution with chemically synthesized miRNAs.

In FIG. 5a, an OR logic gate is designed to recognize two oncogenic miRNAs, which are biomarker miRNAs for HCC. In the presence of either miRNA or both miRNAs, the gate gave output 1. In conclusion, OR gate gives TRUE for two oncogenic miRNAs.

In FIG. 5b, a NAND logic gate is designed to recognize two tumor suppressor miRNAs, which are also biomarker miRNAs for HCC. In the absence/presence of either miRNA or in the absence of both miRNAs, the gate gave output 1. However, in the presence of both tumor suppressor miRNAs, the gate gave output 0. In conclusion, NAND logic gate gives FALSE for two tumor suppressor miRNAs, meaning the answer to the question “Are these miRNA expressions cancerous?” is FALSE when both tumor suppressor miRNAs are present, but TRUE when only one tumor suppressor miRNA is present.

Lastly, an IMPLY logic gate comprising a YES modular logic unit for an oncogenic miRNA and a NOT modular logic unit for a tumor suppressor miRNA was set. In the presence of oncogenic miRNA, the gate gave output 1, and in the absence of both oncogenic miRNA and tumor suppressor miRNA, the gate gave output 1. However, in the absence of oncogenic miRNA and in the presence of tumor suppressor miRNA, the gate gave output 0. In conclusion, IMPLY TRUE output is given for the presence of oncogenic miRNA, and IMPLY FLASE is given when only tumor suppressor miRNA is present.

Further, a kit for detecting oligonucleotides can be contemplated for disease diagnosis, which comprises molecular beacons and at least one DNA logic gate selected from the OR gate, NAND gate, and IMPLY gate. When more than one DNA logic gate is to be used, the molecular beacons have the same fluorophore or different fluorophores for each gate. If there are a smaller number of DNA modular logic units than the number of DNA modular logic units that a DNA board can integrate, the kit may comprise pairs of blocking strands, which are single stranded DNAs to cover the rail sequences not hybridizing to a DNA modular logic unit.

The disease to be diagnosed with the kit is one selected from neurodegenerative diseases, metabolic diseases, cardiovascular/renal/pulmonary diseases, immune diseases, and cancer, and in this case the disease is cancer, in particular hepatocellular carcinoma. The kit may optionally comprise a non-disease control sample, for example miRNA extracted from normal cells.

For the application of the DNA logic gate to real samples, a method comprising steps of dropping a certain amount of the OR, NAND, or IMPLY logic gate in a pH buffer solution to a certain volume of a liquid sample (e.g., blood, serum, saliva, mucus, urine, and other body fluids) or dropping a certain volume of a liquid sample to a substrate coated with certain amount of OR, NAND, or IMPLY logic gate, and waiting at room temperature for 5-30 minutes for fluorescence detection. The substrate to be coated with those gate complexes can be paper, nitrocellulose or polyvinylidene fluoride (PVDF) membrane, or porous or non-porous nitrocellulose film-coated glass or resin.

A kit for education to help understanding DNA logic gates, comprising at least two different miRNAs in solution, OR gate, NAND gate, and IMPLY gate in individual solutions, and a flashlight can also be contemplated.

### EXAMPLES

### Example 1. Materials and Methods

### 1.1. Materials

DNase/protease-free water was purchased from Fisher Scientific Inc. (Pittsburgh, PA, USA) and used for all buffers and oligonucleotide stock solutions. MgCl₂ (1 M solution) was purchased from Thermo Scientific (Waltham, MA, USA), 1M Tris-HCl pH 7.4 buffer from KD Medical (Columbia, MD, USA), and Triton X-100 from Sigma-Aldrich (Burlington, MA, USA). All oligonucleotides were custom-made by Integrated DNA Technologies, Inc. (Coralville, IA, USA), and their stock solutions were prepared by resuspension in water and stored at −20° C. until use. The concentrations of the oligonucleotide stocks were determined using the Beer-Lambert equation, for which absorbance at 260 nm was measured with a Thermo Scientific Nanodrop One UV-Vis Spectrophotometer, while the corresponding extinction coefficients were determined using OligoAnalyzer 3.1 software (Integrated DNA Technologies, Inc.) (Table 1). Fluorescence assays were performed with Perkin Elmer LS 55 Fluorescence Spectrometer (Waltham, MA, USA), Deuterium Lamp. Gel electrophoresis experiments were performed using BioRad electrophoresis equipment (Hercules, CA, USA), and visualized using BioRad Gel Doc XR+.

### 1.2. DNA Logic Gate Assembly

The DNA computing nanostructure is composed of two functionally distinct components (i) a DNA board, and (ii) modular DNA logic units. The in silico analysis of each structural component was performed using the DINAMelt: two-state melting hybridization and Quickfold applications under the UNAfold Web Server. The parameters of the analysis were set considering experimental conditions (22° C., 0.01 M NaCl, 0.05 M MgCl₂, and 0.1 μM oligonucleotides).

All DNA oligonucleotides were mixed at 200 nM in equimolar ratios in a buffer mix containing 100 mM Tris-HCl at pH 7.4, 100 mM MgCl₂, and 0.06% Triton X-100, followed by vortexing and centrifugation to make sure all the solution was dragged down. The samples were annealed by heating the mixture solution up to ˜95-98° C. for 2-5 min and slowly cooling down to room temperature ˜22-25° C. within 8 h.

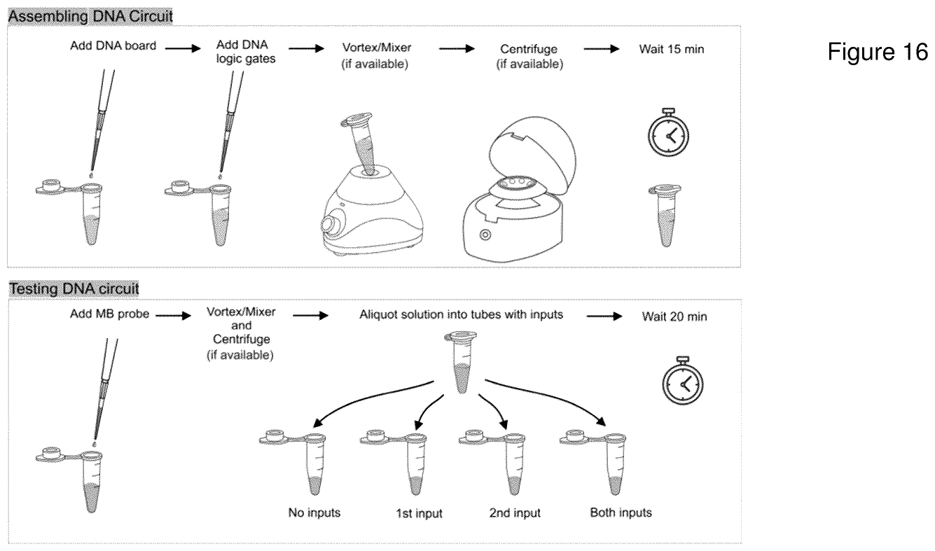

FIG. 16 provides an overview of the experimental steps; to assemble three DNA logic gates (OR, IMPLY, and NAND) by mixing premade stock solutions of the DNA board and DNA modular logic units. To build the three DNA logic gates, a total of 15 different solutions are needed: (1) DNA grade water, (2) DNA board, (3-5) each YES modular logic unit, (6, 7) each NOT modular logic unit, (8, 9) each MB probe, and (10-15) each input (FIG. 16A,C). Step 1. Assembling the DNA logic circuits: In a clean microcentrifuge tube labeled with the name of the DNA circuit, students mixed 300.0 μL of DNA board solution and the volume of DNA logic gates (YES and/or NOT) as specified in the student manual and incubated the mixture for 15 min at room temperature (22-25° C.) to allow molecular assembly. Step 2. Testing the DNA logic circuits: After the 15 min incubation from Step 1, proceeding to test the DNA circuits by adding 15.0 μL of the specified MB stock solution into the assembled DNA circuit, mixing, and then dispensing 40.0 μL of this mixture into four new microcentrifuge tubes, each holding one of the four possible input combinations. Then, the fluorescence signal was observed/measured after incubating the mixtures at room temperature (22-25° C.) for 20 min.

### 1.3. Fluorescence Assays

After assembly, a master mix solution was prepared containing molecular beacon (MB) probe solution and the DNA assembly. From this master mix, aliquots were dispensed in individual microcentrifuge tubes for the addition of the different inputs, followed by incubation at room temperature (22-25° C.) for 20 min. The fluorescence emission was read from those samples, containing 100 nM DNA logic gate assembly, 50 nM MB probe (12.5 nM for YES 1 and IMPLY), 100-200 nM input, 50 mM Tris-HCl at pH 7.4, 50 mM MgCl₂, 0.03% Triton X-100.

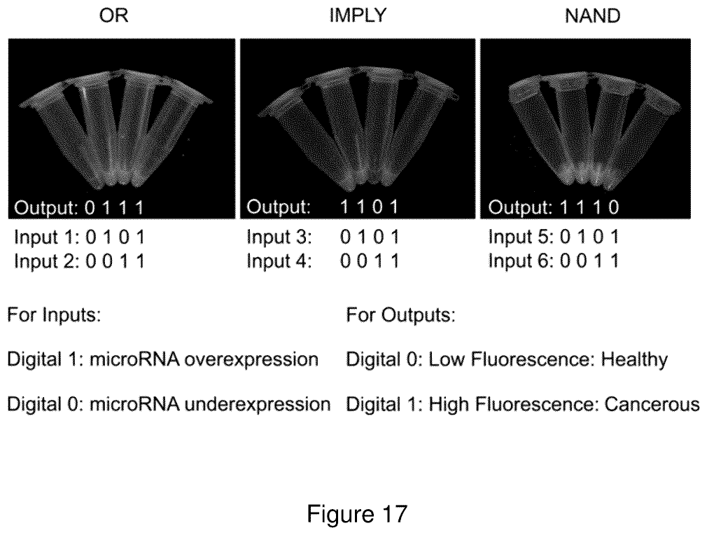

If choosing a spectrofluorometer, excitation and emission, wavelengths must be set at 485 and 517 nm, respectively. Spectrofluorometers provide a quantitative value, where digital output 0 is at least 3-fold lower than digital output 1. If using a blue LED flashlight, anti-UV/blue light safety goggles are needed to block the blue light background and visually observe the emitted fluorescence from the samples. This option provides a qualitative measurement, where digital output 0 shows a distinguishably lower fluorescence intensity than digital output 1, as shown in FIG. 6. The readout corresponding to each input combination (no inputs, each of the two inputs, or both inputs together) allowed its interpretation as either cancerous or healthy states (FIG. 17). The output pattern for each gate was analyzed using the corresponding Boolean truth table to determine whether overexpression (digital 1) or underexpression (digital 0) of the specific miR input triggered the cancerous output (FIG. 17). It was concluded that cancerous conditions are associated with overexpression of either Input 1 or 2 based on the OR output pattern. From the NAND output pattern, it can be concluded that underexpression of either Input 5 or 6 would indicate cancer. Correspondingly, simultaneous underexpression of Input 3 and overexpression of Input 4 are required to maintain a healthy condition (FIG. 6, IMPLY gate output).

### 1.4. Fluorescence Data Analysis

Average and standard deviations were calculated from three independent samples. To normalize the fluorescence response of each output signal, the average fluorescence response of a MB-only solution was subtracted. Each graph plots the average fluorescence difference (ΔF): fluorescence output signal—fluorescence MB signal. Error bars represent the standard deviation from three independent samples.

### 1.5. Gel Electrophoresis

Native gels were prepared with 8% acrylamide (19:1 acrylamide/bisacrylamide) and contained 50 mM MgCl₂. Gels were run at constant voltage (95 V) for 75 min. Samples were prepared using a 6× Cyan/Yellow loading buffer (TrackIt™, Thermofisher, Waltham, MA, USA). TBE buffer (89 mM Tris Base, 89 mM boric acid, and 2 mM EDTA) was used as the running buffer. Denaturing gels were prepared to contain 8 M urea and 12% acrylamide (19:1 acrylamide/bisacrylamide). Samples were prepared using a 2× denaturing loading buffer (85% formamide, TBE, and traces of Bromophenol blue and Xylene Cyanol). Gels were run at constant voltage (150 V) and 65° C. for 1 h and 30 min. Gel-Red was used as a staining dye for the visualization of DNA bands.

### 1.6. Assembly Gel-Extraction: 1

Next, 150 pmol of the DNA assembly was loaded into a native gel. For gel extraction, gels were run at constant voltage (100 V) and 22° C. for 1 h 30 min. The target band was identified and cut with a scalpel blade, followed by being thinly crushed, soaked in 1 mL of DNA-grade water, and incubated under shaking (120 rpm) at 37° C. for up to 24 h. The supernatant was filtered using a X-Spin Coastar filter. From the collected supernatant, DNA was precipitated by adding a 2-fold volume 2% LiClO₄-acetone solution and separated from the supernatant by centrifugation at 10,000 RPM for 3 min (step repeated with pure acetone). The DNA pellet was dried under vacuum for 30-60 min and then resuspended with DNA-grade water.

TABLE 1
Oligonucleotides used in this study

| | | |
|---|---|---|
| Name | comments | Sequence |

DNA Board

| | |
|---|---|
| Rail 1 | CCT ATC GTG TT TTG TCG CTGA CCA TC GTA TCG CTT |
| | CGT CTATG |
| Rail 2 | CTGAG TGAAT GAG CT CTA CA C TGC AGT ACC AC CGT |
| | TAGTCA |
| Staple 1 | ATTCA CTCAG/iSp18//iSp18/ CATAG ACG AAG |
| Staple 2 | GACA AA CAC GAT AGG/iSp18//iSp18/TGA CTA ACG GT |
| | CCAG |
| Blck A₁ | CGA TAC GAT GG |
| Blck B₁ | TGT AGA GCTC |
| Blck A₂ | TCAG CGA CAA |
| Blck B₂ | GGT ACT GCA G |

YES 1

| | |
|---|---|
| A₁ | CT TTG TTC/iSp18/A GAC AAT GTA GC/iSp18/CGATAC |
| | GATGG |
| B₁ | AGTAG AGCTC/iSp18/GAAAC CCA GC/iSp18/GAT G ATT |
| | CC |

NOT 2

| | |
|---|---|
| A₂ | TA CAT TGTC T/iSp18/GGT GAAC C/iSp18/TCAG CGA CAA |
| B₂ | TG TTG CTC/iSp18/GCT GGG |
| Bridge | AGGG GTT CAC CGA GCA ACA TTC/iSp9/GGT ACT GCA |
| | G |

NOT 3

| | |
|---|---|
| A₃ | CT TTG TTC/iSp18/A GAC AAT G/iSp18/CGATAC GATGG |
| | /iSp18/ GC TAC ATT GTCT GC TGG GTTTC |
| B₃ | AGTAG AGCTC/iSp18/AAC CCA GC/iSp18/GAT G ATT CC |

Inputs

| | | |
|---|---|---|
| Input 3 | | GAAAC CCA GC AGAC AAT GTA GC |
| Input 2 | hsa-miR- | /5′ Phos/-rGrArA rUrGrU rUrGrC rUrCrG |
| | 409-3p | rGrUrG rArArC rCrCrC rU |
| Input 1 | hsa-miR- | /5′-Phos/rArGrC rUrArC rArUrU rGrUrC rUrGrC |
| | 221-3p | rUrGrG rGrUrUrUrC |

MB

| | |
|---|---|
| MB₁ | /56-TAMN/CCT GG AATCATC GAACAAAG CA CAG CCAGG |
| | -3′-BHQ2 |
| MB₂ | /56-FAM/CCAGG CCCAGC AGACAATGTA CCT GG/3BHQ_1/ |

Sequences of the same colour in different strands are complementary to each other; the colour code corresponds to that shown in Figures. Italic sequences indicate MB complementarity; underlined sequences, gate connectivity; bold sequences, input complementarity. Each sequence is entered as 5′->3′; iSp9 and iSp18 are oligoethylene glycol spacers 9 and 18 from IDT; /5Phos/, 5′ terminal phosphate group; r indicates ribonucleotide.

### Example 2. DNA Board

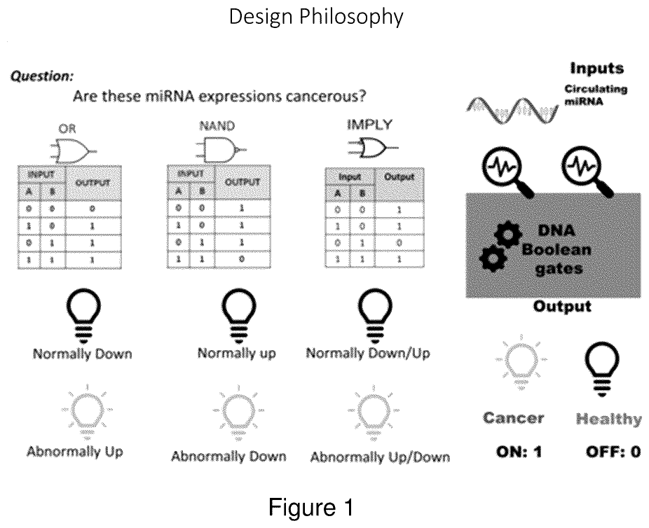

The DNA board is an assembly of four strands-two rails and two staples. Staple 1 and Staple 2 are bound to terminal nucleotides of Rail 1 and Rail 2 by forming 10 to 15 base pairs (FIG. 1 and FIG. 4), leaving single-stranded regions in the middle of both rails for accommodating modular logic units. This single-stranded region serves as a flexible hybridization board for integrating multiple modular logic units, which allows for building a DNA circuit. Sequences and lengths of the nucleotides for base-pairing between the staples and rails were selected considering free energy (ΔG) lower than −12.0 kcal/mol and melting temperature (Tm) higher than 30° C. The length of the ssDNA region in the DNA board can vary according to the number of logic units to be inserted (FIG. 4). Additionally, length of the DNA board can be extended by connecting two homologous DNA boards with oligonucleotides (length 20-40 n.t.) complementary to the staple binding sequences of the rails (FIG. 4).

### Example 3. DNA Modular Logic Units (Gates)

The second component of the DNA computing nanostructure is at least one DNA modular logic unit (gate), a YES modular logic unit and/or a NOT modular logic unit.

A YES modular logic unit consists of two cooperative strands (A and B) (FIG. 2). Each of A strand and B strand is categorized by five regions: scaffold/board binding region, linker, input binding region, another linker, and output forming region. Both linkers can be either oligonucleotides (10-20 nucleotides), non-nucleic acid of 10-20 nucleotide length, or combination thereof.

A NOT modular logic unit can be composed of two strands (A and B) or three strands (A, B′ and C). First, in a NOT modular logic unit, the input/analyte binding region of a YES modular logic unit binds to an auxiliary strand, a single stranded DNA of 10-200 nucleotides. Second, the input/analyte binding region is in the auxiliary strand. Third, one strand of the modular logic unit has an extension of a linker and the auxiliary strand, which forms a bridge strand connected to a board hybridization region. The linker between the auxiliary strand and board hybridization region can be either oligonucleotide (5-10 nucleotides), non-nucleic acid of 5-10 nucleotide length, or combination thereof.

In a two strand NOT modular logic unit, one strand, either A or B, has a bridge strand. In a three strand NOT modular logic unit, instead of one B strand, it has a B′ strand, which does not have a board hybridization region nor a bridge strand, and C strand, which has the board hybridization region and the bridge strand.

The board hybridization regions are designed to tightly bind to the DNA board. Each logic unit binds to the ssDNA regions of both rails by forming 10 to 11 base pairs, which corresponds to one helical turn of B-DNA. This base pair length is applied to all modular logic units to make them align in the same orientation on a 3D plane (FIG. 4); otherwise, their communication efficiency can be compromised. Each logic unit is localized on the rail consecutively, and there is no spacing on the rails between each modular logic unit. Sequences of scaffold binding regions of logic units were selected considering free energy (ΔG) lower than −12.0 kcal/mol and melting temperature (Tm) higher than 35° C.

The input binding region binds to a complementary DNA or RNA input analyte. The first output binding region binds to a molecular beacon reporter. Other output binding regions bind to the input binding region (in a YES modular logic unit) or the auxiliary strand binding region (in a NOT modular logic unit) of the preceding modular logic unit to communicate with the first modular logic unit (FIG. 5 and FIG. 6).

The length and sequence identity of input and output binding regions are customizable according to the analytes of interest and molecular beacon reporters to be used. This gives the advantage of using DNA modular logic units that can be programmed for various logic operators, which in electronic computers is known as universality. Such capability can be applied to low-cost manufacturing and scaling of DNA molecular computers.

### Example 4. DNA Logic Gate Test

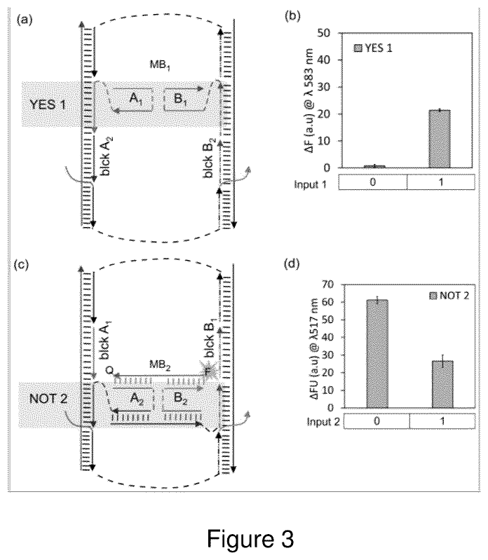

First, in order to experimentally show this programming universality, two DNA modular logic units mimicking YES and NOT logic were designed (FIG. 3). Next, to show the potential that this newly designed YES and NOT logic modular logic units have as building blocks for the construction of DNA circuits with sensing capabilities, the integration of two elemental logic gates were designed to build up a two-layer DNA logic OR (YES-YES), NAND (NOT-NOT), and IMPLY (YES-NOT) gates (FIG. 5 and FIG. 6).

To test the response of each of the logic gates, microRNAs were used as analytes. Five chemically synthesized miRNAs associated with hepatocellular carcinoma (HCC) were selected as inputs and their performance tested in solution. The selected miRNAs are biomarkers associated with hepatocellular carcinoma (a common type of liver cancer), whose expression levels can be increased or decreased in such a disease. Therefore, the invention was challenged to answer whether the sample is related to liver cancer or not in correlation to the concentration of microRNAs tested.

Each of these two-layer logic gates aims to respond as TRUE or FALSE to the question. As an example, a question was asked: are these miRNA expressions cancerous? For instance, OR gate gives False (0) if two oncogenic miRNAs are absent, NAND gate gives False (0) if two tumor suppressor miRNAs present, and lastly, IMPLY gate gives False (0) output if an oncogenic miRNA is absent and one tumor suppressor miRNA is present. Therefore, each of these 2-bit logic gates can concordantly give the same response even when opposite miRNA expression levels are encountered. It can be foreseen that all three gates can be simultaneously used within the same sample.

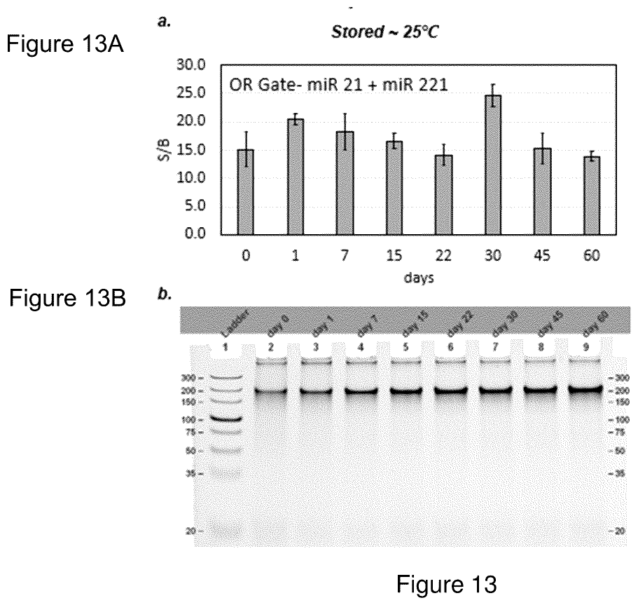

Overall, under experimental conditions, the logic operators followed the expected response when adding synthetic microRNA analytes, and performed its designed logic computations in aqueous solution at room temperature ˜22-25° C. This DNA nanostructure showed a robust response after two months when stored in the same conditions (FIG. 13). Thus, it can be manufactured and stored before shipment and usage. Furthermore, the results show the invention is suitable to integrate the different concentration levels of the analytes and agree in diagnosis.

In conclusion, the invention disclosed here showed that (i) each DNA gate proposed followed its Boolean truth table (FIG. 5a) (ii) Their assembly is traceable through gel electrophoresis (OR gate, FIG. 5b), (iii) they can be stored in solution at ˜25° C. without losing gate integrity over a period of 2 months (FIG. 5c).

### Example 5. IMPLY Logic Circuit (YES+NOT)

First, the performance of individual YES 1 and NOT 2 was optimized on the DNA board structure to achieve the correct digital response. Input 1 and Input 2 are the DNA sequences corresponding to has-miR-221-3p and hsa-miR-409-3p, respectively. Input 1 is recognized by YES 1, while Input 2 is recognized by NOT 2 (Table 1). Upon input recognition, YES 1 combines A1 and B1, giving an output sequence of a total of 18 nucleotides (nt) long. Conversely, NOT 2 dissociates its output sequence (17 nt) upon input recognition by the bridge strand. When only YES 1 was assembled on the DNA board, blocker strands blck A₂ and blck B₂ were added to cover the empty ssDNA regions on both Rail strands (FIG. 3a). Similarly, when only NOT 2 was assembled on the DNA board, blck A₁ and B₁ were added (FIG. 3c). Signal enhancement for YES 1 and signal reduction for NOT 2 was observed, as expected, in the presence of the input strand (FIG. 3b,d).

Then, both YES 1 and NOT 2 gates were integrated on the DNA board such that the output of NOT 2 served as an input for YES 1, as shown in FIG. 8a. In this arrangement, the system was expected to perform as a two-input IMPLY logic gate producing high output (measured as high fluorescence of the MB₁ probe) in all input combinations except when only Input 2 complementary to NOT 2 gate was present (FIG. 8d). The fluorescence assays show the correct digital response of the IMPLY gate (FIG. 8c). An experimental threshold (red dash line in FIG. 8c left) for the differentiation of the ON (digital 1) and OFF (digital 0) output signal of the IMPLY unit was established following the concept of the limit of detection and corresponded to the average signal of YES 1—output 0 plus three times its standard deviation (SD).

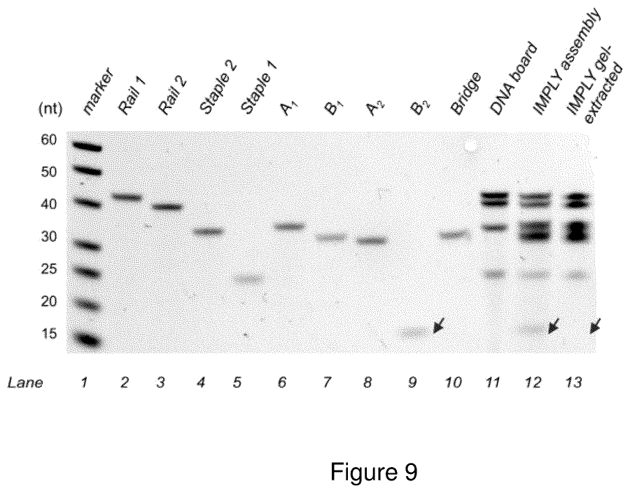

Also the full assembly of the YES 1 and NOT 2 gates on the DNA board was assessed through gel electrophoresis (FIG. 8b). Lane 4 shows faster mobility of the IMPLY unit than that of the DNA board alone (Lane 3). This can be explained by the higher overall negative charge of the ‘loaded’ DNA board nanostructure, which has a comparable electrodynamic volume with that of the unloaded DNA board. To prove that the major band in Lane 4 contained all the expected strands, this band was cut out of the gel, eluted its content, and analyzed the content using denaturing gel electrophoresis (FIG. 9). For mobility reference, individual ssDNA components were added from Lane 2 to 10. Lane 11 shows the four DNA bands corresponding to the mobility of the DNA board components: Rail 1, Rail 2, Staple 1, and Staple 2. The IMPLY full assembly was loaded to Lane 12, which shows six DNA bands corresponding to the overlapping mobility of the components of the DNA board, YES 1 (A₁+B₁) and NOT 2 (A₂+B₂+bridge). The IMPLY assembly after gel extraction was loaded in Lane 13, which shows five DNA bands corresponding to the components of the DNA board, YES 1 and NOT 2's A₂ and bridge. B₂ is not observed in Lane 13 (FIG. 9, blue arrowhead), and since this strand is detached from the DNA board, we consider that under non-equilibrium conditions like those of gel electrophoresis, B₂ is prone to dissociation from the major assembly and was lost from the IMPLY full assembly during gel extraction.

### Example 6. NAND Logic Circuit (NOT+NOT)

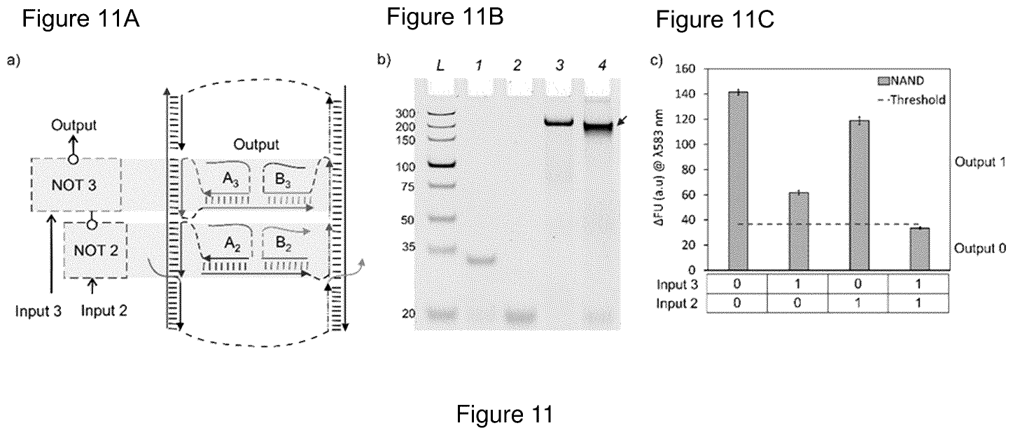

To create a universal NAND function, the DNA board was loaded with two NOT modular logic units (NOT 2+NOT 3) (FIG. 11). NOT 3 recognizes Input 3 (a 22 nt long ssDNA). For later connectivity with NOT 2, NOT 3 was designed to assemble in the same ssDNA region as YES 1 on the DNA board. Additionally, the NOT 3 output sequence is also recognized by MB₁. To test the individual response of NOT 3 on the DNA board, blck A₂ and blck B₂ were added as replacements for NOT 2 strands (A₂ and B₂) to maintain the rigidity of the DNA board. NOT 3 alone showed a 3-fold reduction when Input 3 was added (FIG. 10), demonstrating the digital NOT behavior of this gate.

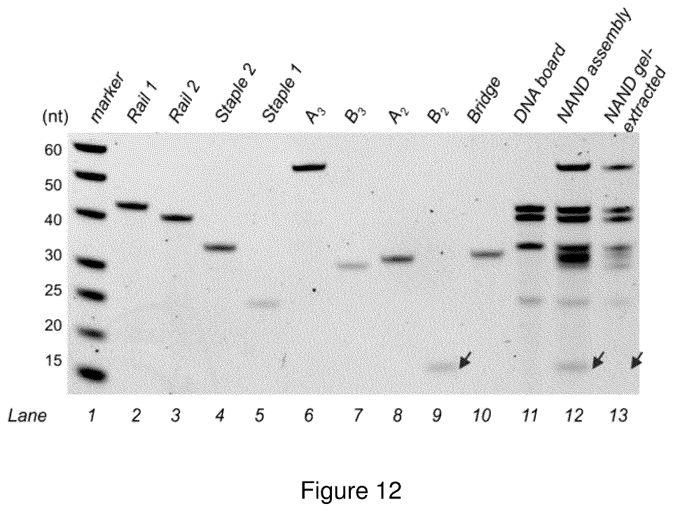

By connecting NOT 3 with NOT 2, a two-input NAND Boolean function was obtained, which is another functionally complete logic gate (FIG. 11a). Similar fluorescence and gel electrophoresis assays as for the IMPLY logic unit were performed. NAND fluorescence assays show the correct digital response as expected based on its truth table (FIG. 11c). Gel electrophoresis also revealed a faster mobility band corresponding to the full NAND assembly (FIG. 11b, Lane 4) as compared with the unloaded DNA board (FIG. 11b, Lane 3). To prove that the major band (shown by a blue arrowhead) in Lane 4 contained all NAND expected strands, a similar procedure as for the IMPLY assembly was performed, by cutting and eluting this band out of the gel and analyzing its content via denaturing gel electrophoresis (FIG. 12).

Denaturing gel electrophoresis (dPAGE) allows for the imaging of the individual constituents of DNA assemblies. The NAND assembly after gel extraction was loaded into Lane 13, which shows seven DNA bands corresponding to the components of the DNA board, NOT 2's A₂ and bridge, and NOT 3. B₂ is not observed in Lane 13 (FIG. 12, blue arrowhead) since this strand is detached from the DNA board. Therefore, in non-equilibrium conditions like those of gel electrophoresis, B₂ is prone to dissociation from the major assembly during gel extraction.

### REFERENCES

[1] A. P. Malvino, J. A. Brown, Digital Computer Electronics, Glencoe McGraw-Hill, 1999.
[2] B. M. Frezza, S. L. Cockroft, M. R. Ghadiri, J. Am. Chem. Soc. 2007, 129, 14875-14879.
[3] A. N. Whitehead, B. Russel, Principia Mathematica, The Syndics Of The Cambridge University Press, 1963.
[4] J. Borghetti, G. S. Snider, P. J. Kuekes, J. J. Yang, D. R. Stewart, R. S. Williams, Nature 2010, 464, 873-876.
[5] M. Prezioso, A. Riminucci, P. Graziosi, I. Bergenti, R. Rakshit, R. Cecchini, A. Vianelli, F. Borgatti, N. Haag, M. Willis, A. J. Drew, W. P. Gillin, V. A. Dediu, Advanced Materials 2013, 25, 534-538.
[6] A. P. D. Silva, Y. Leydet, C. Lincheneau, N. D. McClenaghan, J. Phys.: Condens. Matter 2006, 18, S1847-S1872.
[7] J. Zhou, M. A. Arugula, J. Halámek, M. Pita, E. Katz, J. Phys. Chem. B 2009, 113, 16065-16070.
[8] E. Katz, Ed., DNA- and RNA-Based Computing Systems, John Wiley & Sons, Weinheim, Germany, 2021.
[9] R. E. Polak, A. J. Keung, Current Opinion in Biotechnology 2023, 81, 102940.
[10] D. Fan, J. Wang, E. Wang, S. Dong, Advanced Science 2020, 7, 2001766.
[11] A. Lake, S. Shang, D. M. Kolpashchikov, Angewandte Chemie International Edition 2010, 49, 4459-4462.
[12] Y. V. Gerasimova, D. M. Kolpashchikov, Angewandte Chemie 2016, 128, 10400-10403.
[13] S. Tyagi, F. R. Kramer, Nat Biotechnol 1996, 14, 303-308.
[14] G. Chatterjee, N. Dalchau, R. A. Muscat, A. Phillips, G. Seelig, Nature Nanotech 2017, 12, 920-927.
[15] R. P. Connelly, E. S. Morozkin, Y. V. Gerasimova, ChemBioChem 2018, 19, 203-206.
[16] Y. Benenson, B. Gil, U. Ben-Dor, R. Adar, E. Shapiro, Nature 2004, 429, 423-429.
[17] D. Wang, Y. Fu, J. Yan, B. Zhao, B. Dai, J. Chao, H. Liu, D. He, Y. Zhang, C. Fan, S. Song, Anal. Chem. 2014, 86, 1932-1936.
[18] M. You, G. Zhu, T. Chen, M. J. Donovan, W. Tan, J. Am. Chem. Soc. 2015, 137, 667-674.
[19] X. Song, J. Reif, ACS Nano 2019, 13, 6256-6268

1. A DNA board comprised of two rail strands and two staple strands,
wherein the two rail strands each comprise a single stranded DNA (ssDNA) optionally of a length of at least 40 nucleotides, or optionally 40-200 nucleotides;
wherein the two staple strands each comprise ssDNA, optionally ssDNA at each end linked by a spacer;
wherein one ssDNA end of a first staple strand hybridizes to the 5′-end of a first rail strand and the other ssDNA end of the first staple strand hybridizes to the 3′-end of a second rail strand; and one ssDNA end of a second staple strand hybridizes to the 3′-end of the first rail strand and the other ssDNA end of the second staple strand hybridizes to the 5′-end of the second rail strand;
wherein the rail strand between the rail-staple hybridization regions is single stranded; and
wherein the single stranded region in the rail strand has at least two nucleotide sequences complementary to other DNAs.

2. The DNA board of claim 1, wherein two boards can be connected to form one board in the presence of a pair of single stranded oligonucleotide fragments, wherein a first oligonucleotide fragment hybridizes to the 3′-end of the first rail strand of a first board and to the 5′-end of the first rail strand of a second board with or without a nucleotide gap between both hybridization regions on the fragment; and a second oligonucleotide fragment hybridizes to the 5′-end of the second rail strand of the first board and to the 3′-end of the second rail strand of the second board with or without a nucleotide gap between both hybridization regions on the fragment.

3. The DNA board of claim 1, wherein more than two boards can be connected to form one board in the presence of more than a pair of single stranded oligonucleotide fragments.

4. An OR DNA logic gate comprising at least two YES DNA modular logic units and a DNA board of claim 1,
wherein the YES modular logic unit comprises a pair of single strands, A strand and B strand,
a. the A strand comprising, from 5′ to 3′ end direction, one half of an output binding region, a first linker, one half of an input binding region, a second linker, and a board hybridization region; and
b. the B strand comprising, from 3′ to 5′ end direction, the other half of an output binding region, a first linker, the other half of an input binding region, a second linker, and a board hybridization region;
wherein the board hybridization region of the A strand hybridizes to a complementary nucleotide sequence in the single-stranded region of one rail strand; and the board hybridization region of the B strand hybridizes to a complementary nucleotide sequence in the single-stranded region of the other rail strand, such that the DNA board serves as a flexible hybridization board for integrating the modular logic unit(s).

5. The OR DNA logic gate of claim 4, wherein when more than one modular logic unit is integrated into the board, they are arranged in a parallel manner, and there is no nucleotide gap between each modular logic unit binding sequence in the single stranded rail of the DNA board.

6. The OR DNA logic gate of claim 4, wherein the output that hybridizes to the output binding region of a first modular logic unit is a molecular beacon.

7. The OR DNA logic gate of claim 4, wherein when more than one modular logic unit is integrated into the DNA board, the output that hybridizes to the output binding region of a modular logic unit other than the first modular logic unit is an input binding region of a preceding modular logic unit, which is designed to be complementary to the output binding region of a subsequent modular logic unit.

8. The OR DNA logic gate of claim 4, wherein in the presence of a molecular beacon and an input (analyte) complementary to the nucleotide sequence of any input binding regions, the first modular logic unit forms a 4-way junction to generate fluorescence signal from the molecular beacon.

9. A NAND DNA logic gate comprising at least two NOT DNA modular logic units and a DNA board of claim 1,
i. wherein the NOT modular logic unit comprises a set of two single strands, A strand and B strand:
a. the A strand comprising, from 5′ to 3′ end direction, one half of an output binding region, a first linker, one half of an auxiliary strand binding region, a second linker and a board hybridization region;
b. the B strand comprising, from 3′ to 5′ end direction, the other half of an output binding region, a first linker, the other half of an auxiliary strand binding region, a second linker, a board hybridization region, and a bridge strand comprising a third linker and an auxiliary strand;
and/or
ii, wherein the modular logic unit comprises a set of three single strands, A strand, B′ strand, and a C strand:
a. the A strand comprising, from 5′ to 3′ end direction, one half of an output binding region, a first linker, one half of an auxiliary strand binding region, a second linker, and a board hybridization region;
b. B′ strand comprises, from 3′ to 5′ end direction, the other half of an output binding region, a first linker, and the other half of an auxiliary strand binding region; and
c. C strand comprises from 5′ to 3′ end direction, a bridge strand comprising an auxiliary strand and a third linker, and a board hybridization region:
wherein the auxiliary strand comprises a single stranded DNA, optionally of a length of 10-200 nucleotides, which comprises a nucleotide sequence complementary to an input (analyte); and
wherein the board hybridization region of the A strand hybridizes to a complementary nucleotide sequence in the single-stranded region of one rail strand; and the board hybridization region of the B strand or C strand hybridizes to a complementary nucleotide sequence in the single-stranded region of the other rail strand, so as that the DNA board serves as a flexible hybridization board for integrating the modular logic unit(s).

10. The NAND DNA logic gate of claim 9, wherein when more than one modular logic unit is integrated into the board, they are arranged in a parallel manner, and there is no nucleotide gap between each modular logic unit binding sequence in the single stranded rail of the DNA board.

11. The NAND DNA logic gate of claim 9, wherein the output that hybridizes to the output binding region of a first modular logic unit is a molecular beacon.

12. The NAND DNA logic gate of claim 9, wherein (i) when more than one modular logic unit is integrated into the DNA board, the output that hybridizes to the output binding region of a modular logic unit other than the first modular logic unit is an auxiliary strand binding region of a preceding modular logic unit, which is designed to be complementary to the output binding region of a subsequent modular logic unit; and/or (ii) in the presence of a molecular beacon and all inputs (analytes) complementary to the nucleotide sequences of all input binding regions on the auxiliary strands, the first modular logic unit fails to form a 4-way junction, and the molecular beacon is released from the gate complex.

13. (canceled)

14. An IMPLY DNA logic gate comprising a YES DNA modular logic unit, a NOT DNA modular logic unit, and a DNA board.

15. The IMPLY DNA logic gate of claim 14, wherein the YES DNA modular logic unit comprises a pair of single strands, A strand and B strand,
a. the A strand comprising, from 5′ to 3′ end direction, one half of an output binding region, a first linker, one half of an input binding region (5-100 nucleotides), a second linker, and a board hybridization region; and
b. the B strand comprising, from 3′ to 5′ end direction, the other half of an output binding region, a first linker, the other half of an input binding region, a second linker, and a board hybridization region;
wherein the board hybridization region of the A strand hybridizes to a complementary nucleotide sequence in the single-stranded region of one rail strand; and the board hybridization region of the B strand hybridizes to a complementary nucleotide sequence in the single-stranded region of the other rail strand.

16. The IMPLY DNA logic gate of claim 14, wherein the NOT DNA modular logic unit comprises:
i. a set of two single strands, A strand and B strand:
a. the A strand comprising, from 5′ to 3′ end direction, one half of an output binding region, a first linker, one half of an auxiliary strand binding region, a second linker, and a board hybridization region;
b. B strand comprises, from 3′ to 5′ end direction, the other half of an output binding region, a first linker, the other half of an auxiliary strand binding region, a second linker, a board hybridization region, and a bridge strand comprising a third linker and an auxiliary strand;
and/or
ii. a set of three single strands, A strand, B′ strand, and a C strand,
a. the A strand comprising, from 5′ to 3′ end direction, one half of an output binding region, a first linker, one half of an auxiliary strand binding region, a second linker, and a board hybridization region;
b. the B′ strand comprising, from 3′ to 5′ end direction, the other half of an output binding region, a first linker, and the other half of an auxiliary strand binding region; and
c. the C strand comprising, from 5′ to 3′ end direction, a bridge strand comprising an auxiliary strand and a third linker, and a board hybridization region;
wherein the auxiliary strand is a single stranded DNA, optionally of a length of 10-200 nucleotides, which comprises a nucleotide sequence complementary to an input (analyte), and
wherein the board hybridization region of the A strand hybridizes to a complementary nucleotide sequence in the single-stranded region of one rail strand (; and the board hybridization region of the B strand or C strand hybridizes to a complementary nucleotide sequence in the single-stranded region of the other rail strand.

17. The IMPLY DNA logic gate of claim 14, wherein (i) the output that hybridizes to the output binding region of a first modular logic unit is a molecular beacon; (ii) a YES DNA modular to logic unit and a NOT DNA modular logic unit alternate in a parallel manner wherein there is no nucleotide gap between each modular logic unit binding sequence in the single stranded rail of the DNA board; (iii) when the first modular logic unit is a YES modular logic unit and the second modular logic unit is a NOT modular logic unit, the output to the NOT modular logic unit is an input binding region of a preceding YES modular logic unit, which is designed to be complementary to the output binding region of a subsequent NOT modular logic unit; (iv) wherein the first modular logic unit is a NOT modular logic unit and the second modular logic unit is a YES modular logic unit, the output to the YES modular logic unit is an auxiliary strand binding region of a preceding NOT modular logic unit, which is designed to be complementary to the output binding region of a subsequent YES modular logic unit; and/or (v) wherein when a molecular beacon is present, in the absence of an input (analyte) complementary to the nucleotide sequence of the input binding regions of the YES modular logic unit and in the presence of an input (analyte) complementary to the nucleotide sequence of the input binding regions of the NOT modular logic unit, the first modular logic unit fails to form a 4-way junction, and the molecular beacon is released from the gate complex.

18. (canceled)

19. (canceled)

20. (canceled)

21. (canceled)

22. (canceled)

23. (canceled)

24. A kit for detecting oligonucleotides for disease diagnosis, the kit comprising molecular beacons and at least one DNA logic gate selected from an OR logic gate, a NAND logic gate, and IMPLY logic gate, wherein each gate has the same fluorophore or different fluorophore.

25. The kit of claim 24, wherein, (i) when there are a smaller number of DNA modular logic units than the number of DNA modular logic units that the DNA board can integrate, the kit comprises pairs of blocking strands, which are single stranded DNAs to cover the rail sequences not hybridizing to a DNA modular logic unit; (ii) the disease to be diagnosed is one selected from neurodegenerative diseases, metabolic diseases, cardiovascular diseases, renal diseases, pulmonary diseases, immune diseases, and cancer; and/or (iii) the DNA logic gate is coated on a substrate selected from paper, nitrocellulose or polyvinylidene fluoride membrane, or porous or non-porous nitrocellulose film-coated glass or resin.

26. (canceled)

27. (canceled)

28. (canceled)

29. (canceled)

30. (canceled)

31. (canceled)

32. A method of using the kit of claim 24 for the diagnosis of disease, the method comprising steps of dropping a certain volume of a liquid sample to a certain amount of OR, a NAND, or an IMPLY logic gate-coated substrate, waiting at room temperature for a predetermined amount of time, and detecting the fluorescence.

33. A kit for education to help understanding of DNA logic gates, comprising at least two different miRNAs in solution, DNA logic gates of the OR gate, NAND gate, and IMPLY gate of any of claim 24 in individual solutions, and a flashlight.
